(19) United States
(12) Patent Application Publication
(10) Pub. No.: US 2020/0048660 A1
(43) Pub. Date: Feb. 13, 2020

(54) BACILLUS MUCILAGINOSUS AND HIGH-DENSITY FERMENTATION METHOD AND USE THEREOF

(71) Applicant: Shanxi Green Biotechnology Industry Co., Ltd, Shanxi (CN)

(72) Inventors: Yaogui MU, Shanxi (CN); Liangqi ZHAO, Shanxi (CN); Wenwang QIN, Shanxi (CN); Lihua LV, Shanxi (CN); Qi CHEN, Shanxi (CN)

(21) Appl. No.: 16/375,011

(22) Filed: Apr. 4, 2019

Related U.S. Application Data
(63) Continuation of application No. 15/324,772, filed on Jul. 11, 2017, now abandoned.

(30) Foreign Application Priority Data
Jul. 9, 2014 (CN) 201410324943.X

(51) Int. Cl. C12P 1/04; C12R 1/07; C12N 1/20; C12N 3/00

(52) U.S. Cl. CPC C12P 1/04; C12R 1/07; C12P 2203/00; C12N 3/00; C12N 1/20

(57) ABSTRACT

This invention discloses a mutated strain of wild type Bacillus mucilaginosus HSCUP-76-8 and a high-density fermentation method thereof. The mutated strain HSCUP-76-8 was assigned an Accession No. CGMCC No. 8481. A two-stage and regulated high-density fermentation method has been established for the production of HSCUP-76-8. In the first stage, parameters are controlled to reduce the viscosity of the fermentation broth and promote the growth of bacteria, thereby allowing the bacteria to reach an amount in the range of 2.0×10⁹ cfu/mL-2.3×10⁹ cfu/ml. In the second stage, nutritional factors and fermentation conditions are controlled to promote sporulation, thereby producing endospores in the range of 1.5×10⁹ cfu/mL-2.0×10⁹ cfu/ml. The fermentation cycle of the two-stage high-density fermentation is 32-48 hours.

### FIELD OF THE INVENTION

This invention is related to Bacillus in particular to Bacillus mucilaginosus. Specifically, this invention is related to a type of Bacillus mucilaginosus, and a high-density fermentation method and uses thereof.

### BACKGROUND OF THE INVENTION

Potash shale, a sedimentary rock enriched in potassium element, is an important mineral raw material for the production of potassium fertilizers. According to geological survey, Taihang mountain and Lvliang mountain are enriched with billions of tons of potash shales. Laboratory testing shows that the ores does not only contain potassium, phosphorus and sulfur, but also microelements including iron, zinc, copper, manganese, molybdenum, boron, selenium, sodium and so on which are essential for the growth of various plants, and the ores are ideal for the production of potassium and phosphorus fertilizers. However, these potassium and phosphorus salts are in the mineralized forms which are immobilized and cannot be directly absorbed or utilized by the agro-plants. These salts have to be converted by relevant microorganisms in order to enter the biological cycle and become green fertilizers that can be directly utilized by the crops.

According to the industry standard of silicate bacterial fertilizer NY413-2000 of the People's Republic of China, the number of living bacteria in a liquid state fertilizer product should be at least 5×10⁸ cfu/ml. Relevant literature reveals that: in the paper titled “The research of fermentation condition of Bacillus mucilaginosus” written by Liu Wu-xing in 2002, the number of fermented endospores of Bacillus mucilaginosus NS01 could reach 6.5×10⁸ cfu/ml or more; in the paper titled “The culture condition and the optimization of fermentation process of jelly-like Bacillus mucilaginosus” written by Wu Xiang-hua in 2006, the number of high density fermented endospores of Bacillus mucilaginosus 100130 was 9.85×10⁸ cfu/m1; in the paper titled “The culture condition and the optimization of fermentation process of Bacillus mucilaginosus 021120” written by Wu Siu-fang in 2007, the number of fermented endospores formation of Bacillus mucilaginosus was 9.8×10⁸ cfu/ml; and in the patent titled “The low viscosity and high productivity fermentative production method of Bacillus mucilaginosus PM13 strain” of Zhao Bing et. al. in 2010, the number of endospores could reach 1.5×10⁹ cfu/ml in maximum, which is the highest number of endospores production up to date reported.

We have conducted numerous researches for many years for the research and development of potash shale bacterial fertilizers. An invention titled “Biological mineral complex fertilizer and its production method” was filed in China in 2009 and has been granted (ZL200910073705.5). While producing the biological mineral complex fertilizer, we have also conducted research about mutation breeding and fermentation of the wild type indigenous bacterial strains selected from the mineral areas, in order to improve the utilization rate of potash shale and efficacy of the fertilizer.

A few strains which showed good performance in decomposing potassium and phosphorus were obtained from the He-shun mining area of the Shanxi Province. We selected one of the best functioning strains for characterization and classification. After the gram staining, endospore staining, capsule staining, flagella staining, electron microscopic observation, physiological and biochemical assays and sequence alignment analysis of 16S rDNA, the strain has been identified as a type of Bacillus mucilaginosus, and named as Bacillus mucilaginosus HSC. However, the number of bacteria and sporulation rate are not completely satisfactory in the subsequent fermentation production trials.

### DETAILED DESCRIPTION OF THE INVENTION

The present invention has two objectives: (1) to provide a type of Bacillus mucilaginosus that is effective in the decomposition of potash shale, and with a higher number of bacteria and sporulation rate; and (2) to provide a technology for the high-density fermentation for the above-mentioned species of Bacillus mucilaginosus.

The invention provides a type of Bacillus mucilaginosus (Accession no.: CGMCC No. 8481). The original strain of the present type of Bacillus mucilaginosus was isolated from the shale cave in the He-shun mining area of Shanxi Province. After gram staining, endospore staining, capsule staining, flagella staining, electron microscopic observation and other physiological and biochemical assays, and finally sequence alignment analysis of 16SrDNA, the strain has been identified as Bacillus mucilaginosus, name as Bacillus mucilaginosus HSC. After the treatment of ultraviolet mutagenesis and plasma mutagenesis, a mutated strain of Bacillus mucilaginosus (Bacillus mucilaginosus HSCUP-76-8) was obtained, and deposited in the China General Microbiological Culture Collection Center on Nov. 26, 2013, and assigned the Accession No. CGMCC No. 8481. The strain has characteristics in favor of production and application, as indicated by its production-related characteristics including high growth rate, low viscosity of fermentation broth, high bacterial concentration, high sporulation rate, short fermentation time and so on, and its long survival time, as well as its high capability in potassium decomposition with regard to its application characteristic.

The invention provides a two-stage fermentation process for Bacillus mucilaginosus.

Details of which are as follows:

I. Activation of the Strain and Culture and Expansion of the Inoculum
(1) Activation of the strain and culture of the slanting inoculum: Inoculate Bacillus mucilaginosus HSCUP-76-8 on a slanting culture medium for activation, with temperature controlled at 29-33° C., and culture for 24-48 hours, thereby obtaining the original slanting inoculum. Then inoculate the original slanting inoculum on a slanting culture medium, and culture said inoculum under the same prescribed conditions, thereby obtaining the slanting inoculum.
(2) Production of shake-flask inoculum: Inoculate the slanting inoculum in a shake-flask medium with 50-5100 ml medium per 250 ml shake-flask, culture it at 29-33° C., rpm 160-220 r/min, for 8-12 hours to obtain the mother inoculum. Then inoculate the mother inoculum in the shake-flask medium and culture it under the same prescribed conditions, thereby obtaining the liquid of shake-flask inoculum.
(3) Culture of fermentation seed: Using seed fermentation medium, with charging coefficient of 0.7-0.8, steam sterilization under 121° C. for 20 minutes, inoculum size of 5%-10%, culture temperature of 29˜33° C., air flow rate of 1.0:0.8˜1.0 (v/v·min), dissolved oxygen saturation DO value controlled at 10%-30% using a speed agitator, pH value controlled at 7.2±0.2, and culture for 8˜12 hours to obtain a liquid inoculum of fermentation seed at log phase.

II. Two-Stage Fermentation Production
(4) First stage (from inoculation to the end of log phase, with the objective of promoting bacterial growth and reproduction, to obtain the possible maximum number of bacteria): using basal fermentation medium, with charging coefficient of 0.7˜0.8, steam sterilization under 121° C. for 20 minutes, inoculum size of 5%-10%. Control parameters: temperature of 29˜33° C., pH value of 7.0˜7.2, air flow rate of 1:0.8˜1.2 (v/v·min), dissolved oxygen saturation DO value controlled at 20%-30% using a speed agitator, and using vegetable oil as antifoam agent to control the foam. Collect samples every 4 hours, then examine the number and the morphology of the bacteria under microscope, and conduct chemical analysis of sugar and ammoniacal nitrogen. Promptly provide feeding according to the examination results, control sugar dose at 2 g/L-3 g/L, and control nitrogen source at 0.5 g/L˜0.1 g/L using NH₄Cl, then stop providing additional nitrogen towards the end of the log phase.
(5) Second stage (from the end of the log phase to the maturation of the endospores, with the objective of sporulation with the possible maximum sporulation rate), control parameters: temperature of 33-37° C., pH value of 7.2-8.9, air flow rate of 1:1.0˜0.8 (v/v·min), dissolved oxygen saturation in DO value controlled at 5%-10% using a speed agitator, then add calcium carbonate to promote sporulation. Stop fermentation, and store the product when endospores occupy the entire visual field under the microscopic examination.

The above-mentioned slanting culture medium, liquid culture medium and seed fermentation medium can be culture media being used for Bacillus mucilaginosus, with a preference of the following list of culture media:

Slanting culture medium: 5 g sucrose, 1 g NaH₂PO₄, 0.5 g MgSO₄.7H₂O, 0.005 g FeCl₃, 0˜0.1 g CaCO₃, 20˜30 g agar, 1000 ml distilled water, pH 7.0˜7.4.

Liquid culture medium: 5˜10 g sucrose, 0.5˜1 g NH₄Cl, 1˜1.5 g NaH₂PO₄, 0.5˜1 g MgSO₄.7H₂O, 0.005 g FeCl₃, 0.1˜0.3 g CaCO₃, 1000 ml distilled water, pH 7.0˜7.4.

Seed fermentation medium: 2˜5 g sucrose, 3˜10 g starch, 1˜2 g (NH₄)₂SO₄/0.5˜1 g NH₄Cl, 1˜2 g yeast extract, 1˜1.5 g NaH₂PO₄, MgSO₄.7H₂O 0.5˜1 g, 0.005 g FeCl₃, 1000 ml distilled water, pH 7.0˜7.4.

Basal Fermentation Medium: 5 g sucrose, 1˜2 g (NH₄)₂SO₄/0.5˜1 g NH₄Cl, 1˜2 g yeast extract, 1˜2 g NaH₂PO₄, 0.5˜1 g MgSO₄.7H₂O, 0.005 g FeCl₃, 1000 ml distilled water, pH 7.0˜7.4; Sucrose can be substituted with corn flour or starch, or a mixture of sucrose and corn flour.

In comparison with the art, the advantages and effects of the present bacterial strain are characterized by its high capability in decomposing potash shale, high growth rate, low viscosity of fermentation broth, high sporulation rate and the capability in undergoing a high density fermentation.

This invention established a two-stage fermentation method for growing bacteria and sporulation. Using the present method, the number of endospores can reach 1.6×10⁹ cfu/ml˜2.0×10⁹ cfu/ml within 30˜48 hours of fermentation time, with a sporulation rate of 75%-83%.

### DETAILED DESCRIPTION OF THE FIGURES

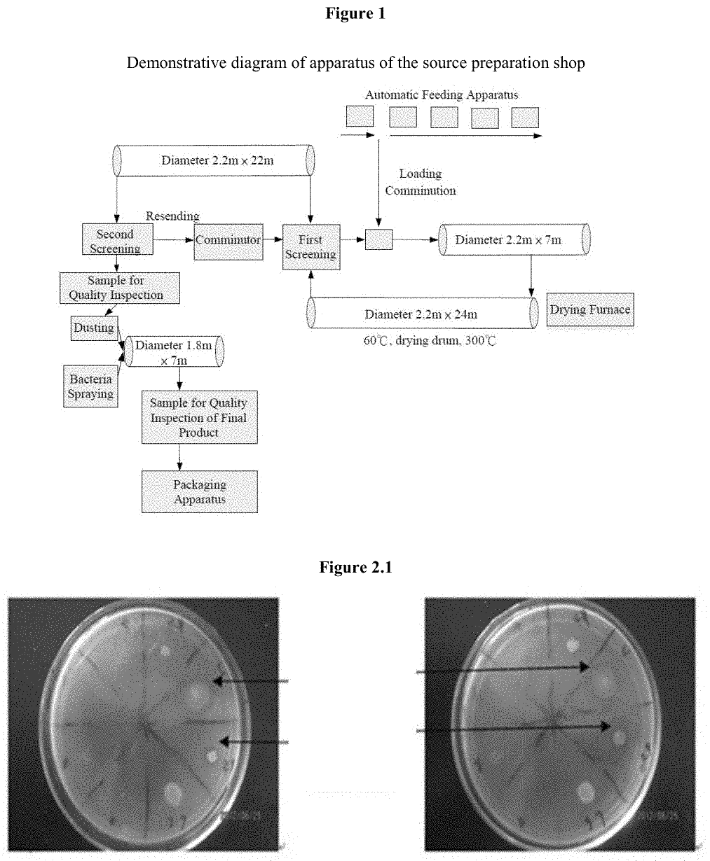

FIG. 1 represents a demonstrative diagram of apparatus of the source preparation shop.

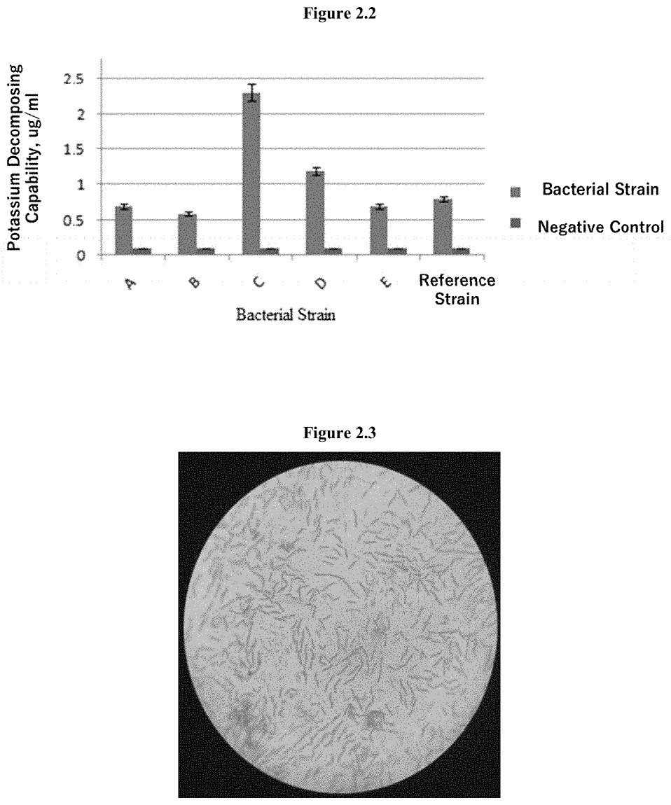

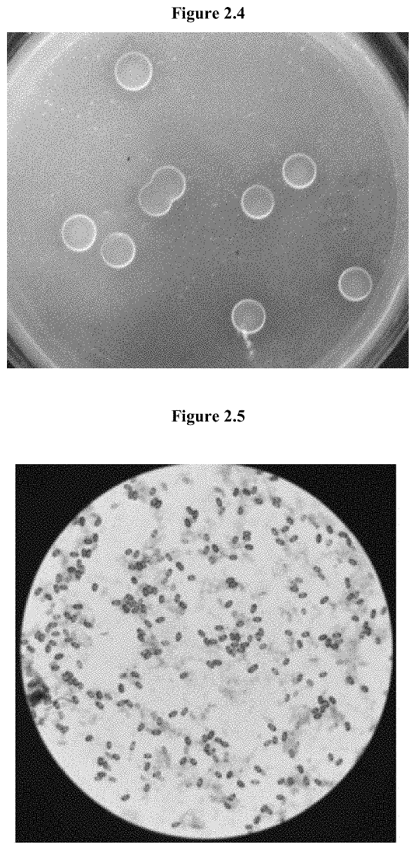

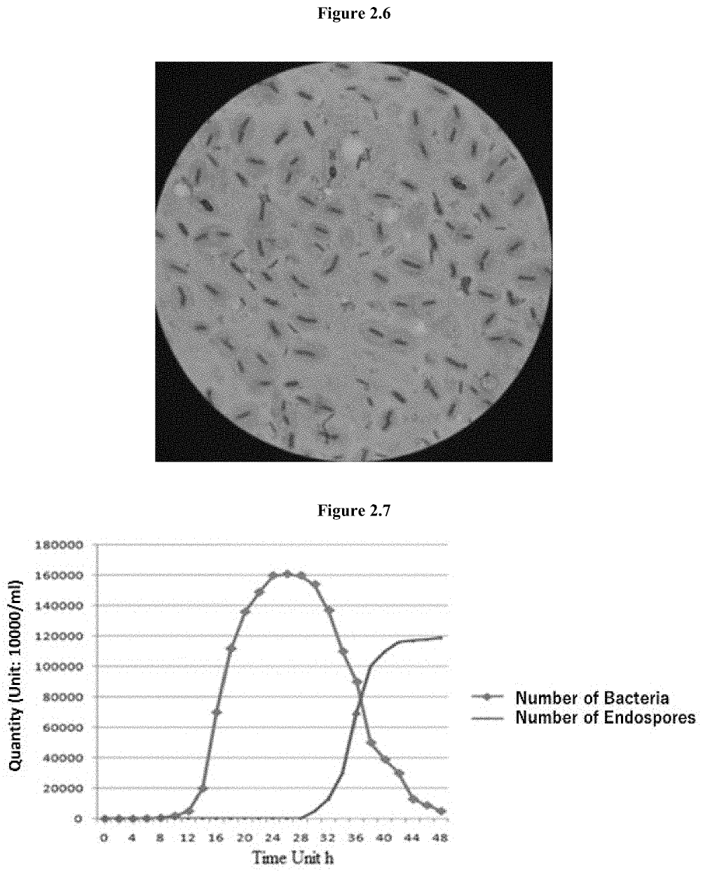

FIG. 2.1 shows the ability of various wild type bacterial strains in decomposing potassium. The medium contains mineral powder such that strains which is capable of decomposing potassium will create a potassium decomposing circle around the colonies. The arrows point at the potassium decomposing bacteria, the central white part is the colonies, and the peripheral transparent part is the potassium decomposing circle.

FIG. 2.2 shows the capabilities of five bacterial strains (A-E) in decomposing potash shale in comparison to a reference strain and a negative control.

FIG. 2.3 shows Bacterium Strain C in the log phase of growth, which was cultured in the medium with starch as carbon source.

FIG. 2.4 shows colonies in a circular form with a uniformed and bulge edge; the colonies are colorless and translucent.

FIG. 2.5 shows endospores having an oval shape, the sporulation site is located at the center or the central of the termini.

FIG. 2.6 shows an enriched capsule produced on a nitrogen-free culture medium.

FIG. 2.7 shows the growth curve and sporulation curve of a batch of fermentation HSC bacteria.

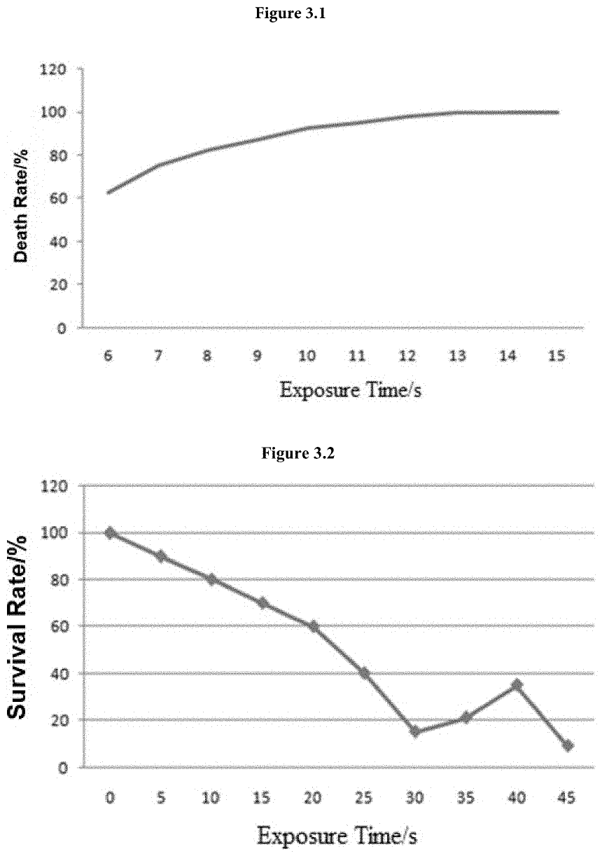

FIG. 3.1 shows the effects of mutagenesis time on the death rate of Bacillus mucilaginosus HSC.

FIG. 3.2 shows the change of survival rate of Bacillus mucilaginosus HSCU-76 upon injection of N+.

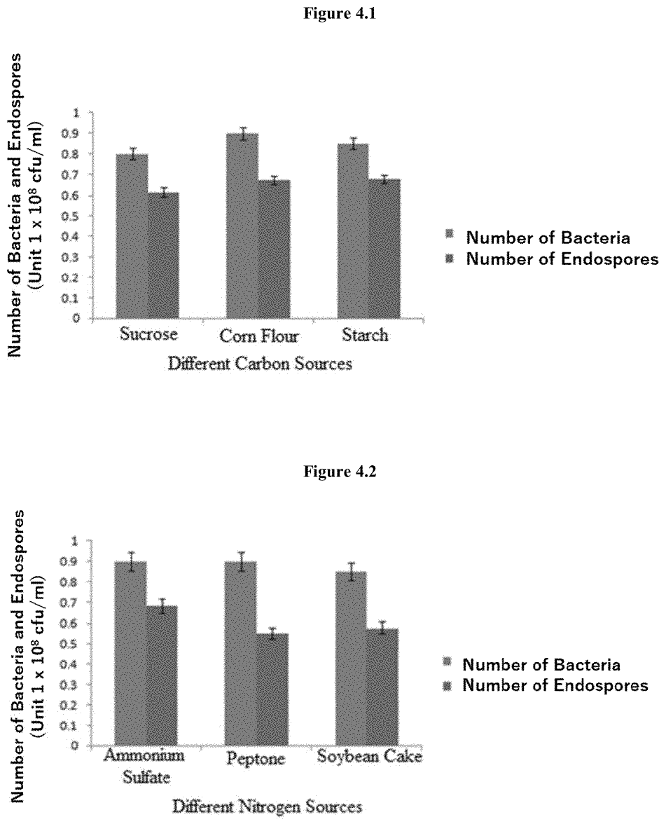

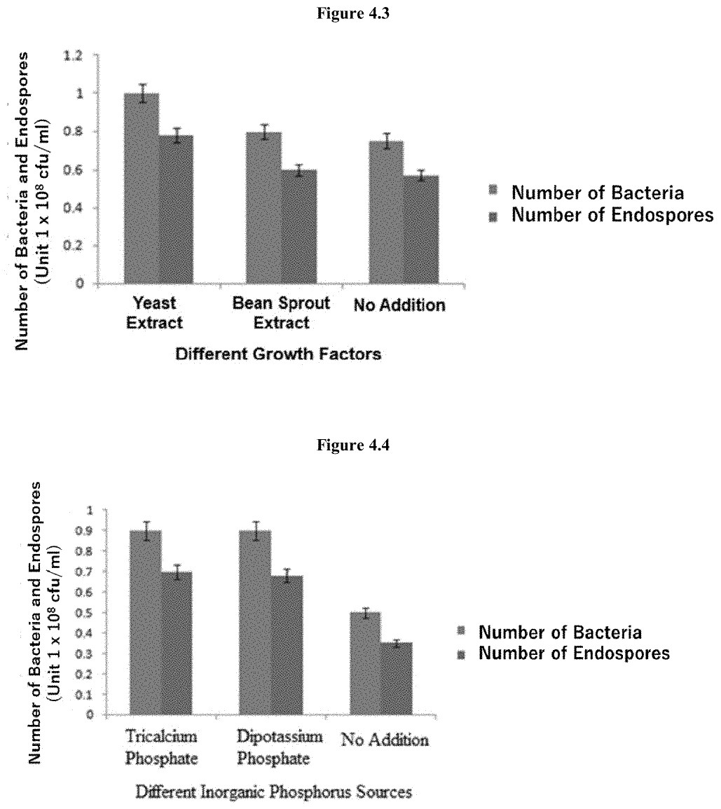

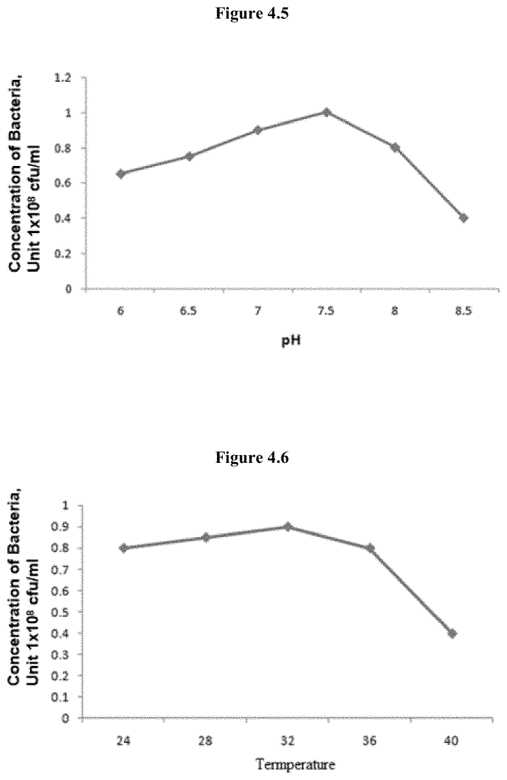

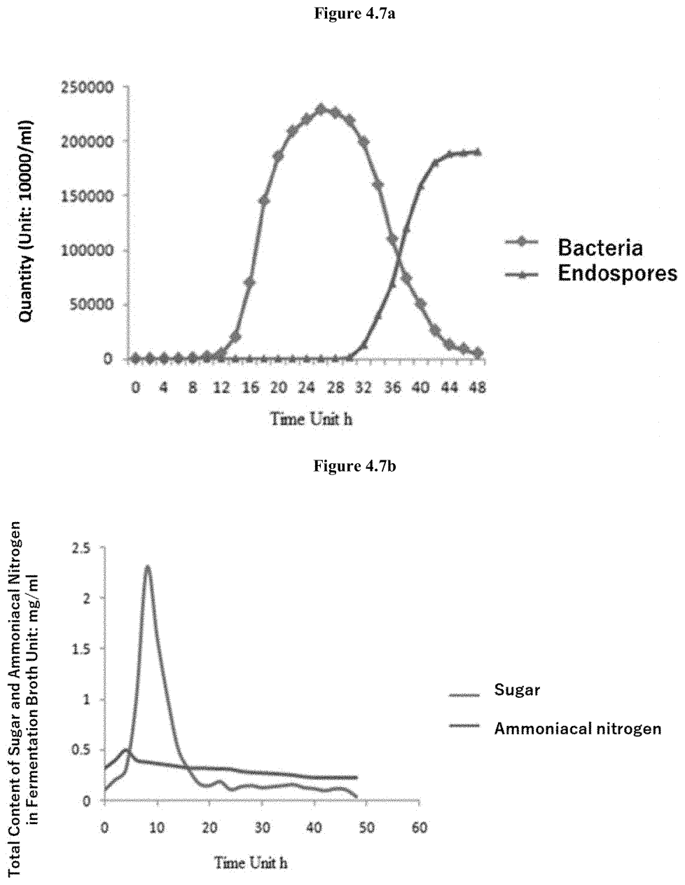

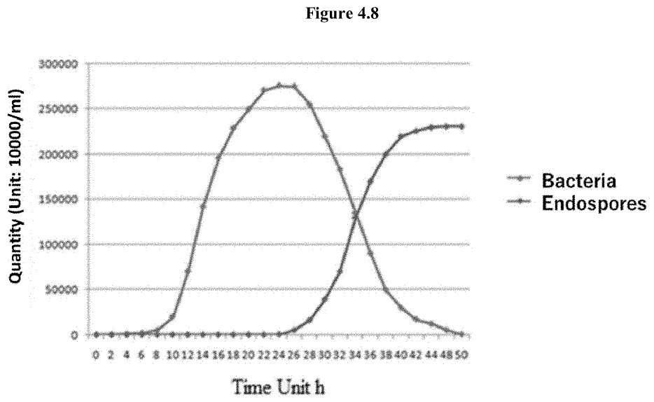

FIG. 4.1 represents the results of a single-factor experiment using sucrose, corn flour or starch as the carbon source.

FIG. 4.2 represents the results of a single-factor experiment using ammonium sulfate, peptone or soybean cake powder as the nitrogen source.

FIG. 4.3 represents the results of a single-factor experiment testing growth factors.

FIG. 4.4 represents the results of a single-factor experiment testing different inorganic phosphorus.

FIG. 4.5 represents the results of a single-factor experiment testing different pH values.

FIG. 4.6 represents the results of a single-factor experiment testing different pH values of FIG. 4.6.

FIG. 4.7a represents a batch fermentation diagram of HSCUP-76-8.

FIG. 4.7b represents the change of the total content of sugar and ammoniacal nitrogen in the fermentation medium during the metabolic process.

FIG. 4.8 shows the bacterial growth curve and sporulation of the two-stage fermentation.

### EXAMPLES

All of the bacterial strains used in the examples are Bacillus mucilaginosus HSCUP-76-8, with an Accession Number CGMCC No. 8481.

The medium used is shown as follows:

Medium 1 (Slanting culture medium): sucrose 5 g, NaH₂PO₄ 1 g, MgSO₄.7H₂O 0.3˜0.7 g, FeCl₃ 0.005 g, CaCO₃ 0.1 g, agar 20 g, distilled water 1000 ml, pH 7.2.

Medium 2 (Liquid culture medium): sucrose 5 g, NaH₂PO₄ 1 g, MgSO₄.7H₂O 0.3˜0.7 g, FeCl₃ 0.005 g, CaCO₃ 0.1 g, distilled water 1000 ml, pH 7.2.

Medium 3 (Seed fermentation medium): sucrose 2 g, starch 3 g, (NH₄)₂SO₄ 1 g, yeast extract 1 g, NaH₂PO₄ 1 g, MgSO₄.7H₂O 0.3˜0.7 g, FeCl₃ 0.005 g, CaCO₃ 0.1 g, distilled water 1000 ml, pH 7.2.

Medium 4 (Basal fermentation medium): sucrose 5 g, (NH₄)₂SO₄ 0.5 g, yeast extract 1 g, NaH₂PO₄ 2 g, MgSO₄.7H₂O 0.3˜0.7 g, FeCl₃ 0.005 g, CaCO₃ 0.1 g, distilled water 1000 ml, pH 7.2.

Medium 5 (Basal fermentation medium): corn flour 5 g, yeast extract 1 g, NaH₂PO₄ 2 g, MgSO₄.7H₂O 0.3˜0.7 g, FeCl₃ 0.005 g, CaCO₃ 0.1 g, distilled water 1000 ml, pH 7.2.

Medium 6 (Basal fermentation medium): starch 5 g, (NH₄)₂SO₄ 0.5 g, yeast extract 1 g, NaH₂PO₄ 2 g, MgSO₄.7H₂O 0.3˜0.7 g, FeCl₃ 0.005 g, CaCO₃ 0.1 g, distilled water 1000 ml, pH 7.2.

Medium 7 (Basal fermentation medium): sucrose 2 g, corn flour 3 g, yeast extract 1 g, NaH₂PO₄ 2 g, MgSO₄.7H₂O 0.3˜0.7 g, FeCl₃ 0.005 g, CaCO₃ 0.1 g, distilled water 1000 ml, pH 7.2.

### Example 1

1. Ultraviolet Mutagenesis
1) Preparation of a suspension of Bacillus mucilaginosus HSC: Bacteria in the log phase of growth were diluted, cells were quantified using haemocytometer, and a concentration of 10⁷˜10⁸ cfu/mL was selected for preparing the original strain, which was then stored for later uses;
2) Treatment with ultraviolet mutagenesis: The bacterial suspension was placed in an aseptic petri dish (diameter 5 cm) which was put under an ultraviolet lamp (power 25 W, and wavelength 254 nm) at a distance of 20 cm and subject to different exposure time (0˜15 minutes). Death rate of the bacteria was estimated by counting cells using a hemocytometer, and the exposure time which led to a 99.9% death rate was selected as the exposure dose (8 minutes). Ultraviolet mutagenesis was then conducted at an exposure dose of 8 minutes, the bacteria was then cultured at a constant temperature of 30° C. for 48 hours. The resulting mutated strains were selected.
3) Initial screening and re-screening: Based on the size of clear zones produced upon decomposition of culture media containing potash shale, 200 mutated strains were selected in the initial screening. The selected mutated strains were re-screened based on the number of colonies in the culture medium, sporulation rate, potassium-decomposing capability and stability of mutated strains; one strain was finally selected and named as Bacillus mucilaginosus HSCUP-76. The sporulation rate of the selected strain can reach 82%.

2. Plasma Mutagenesis
1) Preparation of bacterial pellicle of Bacillus mucilaginosus HSCU-76: 1 ml of bacteria in the log phase of growth was spread on a petri dish, dried aseptically and stored under dark thereafter.
2) Treatment with plasma mutagenesis: Bacterial pellicle was subject to a radiofrequency power source of a cold plasma-modifying device (Type HD), using low-energy N ions as the implantation ions. Under a power of 50 W, plasma mutagenesis was performed for 0 s, 5 s, 10 s, 15 s, 20 s, 25 s, 30 s, 35 s, 40 s and 45 s respectively. Mutated strains were selected.
3) Initial screening and re-screening: Based on the size of clear zones produced upon decomposition of culture media containing potash shale, 30 mutated strains were selected in the initial screening. The selected mutated strains were re-screened based on the number of colonies in the culture medium, sporulation rate, potassium-decomposing capability and stability of mutated strains; one strain was finally selected and named as Bacillus mucilaginosus HSCUP-76-8. The characteristics of the selected strain are as follows: the number of bacteria in the fermentation broth of 2.0×10⁹, the sporulation rate of 82%, potassium-decomposing capability of 2.6 μg/mL, and showing a genetic stability.

### Example 2

1. Activation of the Strain and Culture and Expansion of the Inoculum
1) Activation of the strain and culture of the slanting inoculum: Bacillus mucilaginosus HSCUP-76-8 was inoculated in slanting culture medium for activation, with temperature controlled at 30° C., and cultured for 36 hours, thereby obtaining the original slanting inoculum. The original slanting inoculum was then inoculated in the slanting culture medium and cultured under the same prescribed conditions, thereby obtaining the slanting inoculum.
2) Production of shake-flask inoculum: The slanting inoculum was inoculated in a shake-flask medium with 100 ml medium per 250 ml shake-flask, cultured at 32° C., rpm 200 r/min for 12 hours, thereby obtaining a mother inoculum. The mother inoculum was inoculated in the shake-flake medium and cultured under the same prescribed conditions, thereby obtaining the liquid of shake-flask inoculum.

2. Assay for Testing Potassium Decomposing Capability

In each of the three 250 ml Erlenmeyer flasks, 0.5 g potassium mineral powder was added to 95 ml of shake-flask medium (potassium-free), the solution was sterilized and inoculated with bacteria at 5%. Two control groups were prepared: Group 1 was not inoculated with any bacteria; Group 2 was inoculated with the potassium-decomposing bacteria obtained from the original bacterial fertilizer (and activated). Culture was performed on a thermostatic shaker at 32° C. under 200 r/min for 151. Each of the resulting culture was centrifuged; 4 ml of supernatant was obtained and thoroughly mixed with an equal volume of LiCl (6 mmol/L) for determining the amount of potassium using a frame spectrometer. The results are as follows: Group 1 contained 0.2 μg/mL of potassium; Group 2 contained 1.1 μg/ml of potassium; and the tested group added with HSCUP-76-8 contained 2.6 μg/mL of potassium.

### Example 3

1. Activation of the Strain and Culture and Expansion of the Inoculum
1) Activation of the strain and culture of the slanting inoculum: Bacillus mucilaginosus HSCUP-76-8 was inoculated in the slanting culture medium for activation, with temperature controlled at 30° C., and cultured for 36 hours, thereby obtaining the original slanting inoculum. The original slanting inoculum was then inoculated in the slanting culture medium and cultured under the same prescribed conditions, thereby obtaining the slanting inoculum.
2) Production of shake-flask inoculum: The slanting inoculum was inoculated in a shake-flask medium with 100 ml medium per 250 ml shake-flask, cultured at 30° C., rpm 200 r/min for 12 hours, thereby obtaining a mother inoculum. The mother inoculum was inoculated in the shake-flake medium and cultured under the same prescribed conditions, thereby obtaining the liquid of shake-flask inoculum.
3) Culture of fermentation seed: Using seed fermentation medium 3, with charging coefficient of 0.7, steam sterilization under 121° C. for 20 minutes, the liquid of shake-flask inoculum was inoculated with an inoculum size of 5% and cultured at 29° C., air flow rate of 1.0:0.8˜1.0 (v/v·min), dissolved oxygen saturation DO value controlled at 10%-30% using a speed agitator, pH value controlled at 7.2±0.2, for 8 hours, thereby obtaining a liquid inoculum of fermentation seed at log phase.

2. Two-Stage Fermentation Production
4) First stage: Control parameters: temperature 29˜33° C., pH value of 7.2±0.2, air flow rate of 1:0.8˜1.2 (v/v·min) during the 0^{th}-19^{th} hours, controlled the dissolved oxygen saturation DO value controlled at 10%˜30% using a speed agitator, and used vegetable oil as antifoam agent to control the foam. Samples were collected every 4 hours, then examined the number and the morphology of the bacteria under microscope, and determined the concentration of sugar with Anthrone reagent, and the content of ammoniacal nitrogen by indophenol blue spectrometry. Feeding was promptly provided according to the examination results, sucrose concentration during the 0^{th}-18^{th} hours was controlled at 2 g/L˜3 g/L, and NH₄Cl was controlled at 0.5 g/L˜0.1 g/L, feeding was stopped towards the end of the log phase. The number of bacteria reached 2.0×10⁹ cfu/mL after 20 hours of fermentation.
5) Second stage (from the end of the log phase to the maturation of the endospores). Control parameters: temperature at 33° C. during 20^{th}-26^{th} hours, pH value in the range of 7.0˜7.5, air flow rate of 1:0.8˜1.0 (v/v·min), controlled the dissolved oxygen saturation DO value at 10%-20% using a speed agitator, then supplemented calcium carbonate at 0.5 g/L. Temperature was then changed to 37° C. and pH value adjusted to 8.0˜8.9, cultured until the end of fermentation. The number of endospores reached 1.6×10⁹ cfu/mL after 32 hours of fermentation. Sporulation rate was 80%.

### Example 4

1. Activation of the Strain and Culture and Expansion of the Inoculum
1) Activation of the strain and culture of the slanting inoculum: Bacillus mucilaginosus HSCUP-76-8 was inoculated in the slanting culture medium for activation, with temperature controlled at 30° C., and cultured for 36 hours, thereby obtaining the original slanting inoculum. The original slanting inoculum was then inoculated in the slanting culture medium and cultured under the same prescribed conditions, thereby obtaining the slanting inoculum.
2) Production of shake-flask inoculum: The slanting inoculum was inoculated in a shake-flask medium with 100 ml medium per 250 ml shake-flask, cultured at 30° C., rpm 200 r/min for 12 hours, thereby obtaining a mother inoculum. The mother inoculum was inoculated in the shake-flake medium and cultured under the same prescribed conditions, thereby obtaining the liquid of shake-flask inoculum.
3) Culture of fermentation seed: Using seed fermentation medium 3, with charging coefficient of 0.7, steam sterilization under 121° C. for 20 minutes, the liquid of shake-flask inoculum was inoculated with an inoculum size of 5%, and cultured at 29° C., air flow rate of 1.0:0.8˜1.2 (v/v·min), dissolved oxygen saturation DO value controlled at 30%-10% using a speed agitator, pH value controlled at 7.2±0.2, for 12 hours, thereby obtaining a liquid inoculum of fermentation seed at log phase.

2. Two-Stage Fermentation Production
4) First stage: Using basal fermentation medium 5, with charging coefficient of 0.7, steam sterilization under 121° C. for 20 minutes. Inoculum size of 8%. Control parameters: temperature 30˜33° C., pH value of 7.2±0.2, air flow rate of 1:0.8˜1.2 (v/v·min), controlled the dissolved oxygen saturation DO value at 10%-30% using a speed agitator, and used vegetable oil as antifoam agent to control the foam. Samples were collected every 4 hours and feeding was provided as needed. The number and the morphology of the bacteria were examined under microscope. During the 0^{th}-28^{th} hours, 1.4 g/L˜1.6 g/L of corn flour was supplemented at each time with reference of the sucrose consumption determined in Example 1. Concentration of NH₄Cl was controlled at 0.5 g/L˜0.1 g/L, feeding was stopped at 28^{th} hour. The number of bacteria reached 2.4×10⁹ cfu/mL after 30 hours of fermentation.
5) Second stage (from the end of the log phase to the maturation of the endospores). Control parameters: temperature at 33° C. during 30^{th}-36^{th} hours, air flow rate of 1:0.8˜1.2 (v/v·min), controlled the dissolved oxygen saturation DO value at 10%˜5% using a speed agitator, pH value in the range of 6.9˜7.5. Supplemented with calcium carbonate at 0.5 g/L. Temperature was then changed to 35° C. and pH value adjusted to 8.0˜8.5. Culture was carried on until the end of fermentation (48 hours). The number of endospores reached 1.9×10⁹ cfu/mL after 48 hours of fermentation. Sporulation rate was 79%.

### Example 5

1. Activation of the Strain and Culture and Expansion of the Inoculum
1) Activation of the strain and culture of the slanting inoculum: Bacillus mucilaginosus HSCUP-76-8 was inoculated in the slanting culture medium for activation, with temperature controlled at 30° C., and cultured for 36 hours, thereby obtaining the original slanting inoculum. The original slanting inoculum was then inoculated in the slanting culture medium and cultured under the same prescribed conditions, thereby obtaining the slanting inoculum.
2) Production of shake-flask inoculum: The slanting inoculum was inoculated in a shake-flask medium with 100 ml medium per 250 ml shake-flask, cultured at 30° C., rpm 200 r/min for 12 hours, thereby obtaining a mother inoculum. The mother inoculum was inoculated in the shake-flake medium and cultured under the same prescribed conditions, thereby obtaining the liquid of shake-flask inoculum.
3) Culture of fermentation seed: Using seed fermentation medium 3, with charging coefficient of 0.7, steam sterilization under 121° C. for 20 minutes, the liquid of shake-flask inoculum was inoculated with an inoculum size of 5%, and cultured at 29° C., air flow rate of 1.0:0.8˜1.2 (v/v·min), dissolved oxygen saturation DO value controlled at 30%-10% using a speed agitator, pH value controlled at 7.2±0.2, for 12 hours, thereby obtaining a liquid inoculum of fermentation seed at log phase.

2. Two-Stage Fermentation Production
4) First stage: Using basal fermentation medium 6, with charging coefficient of 0.7, steam sterilization under 121° C. for 20 minutes. Inoculum size of 7%. Control parameters: temperature 32˜33° C., pH value of 7.2±0.2, air flow rate of 1:0.8˜1.2 (v/v·min), controlled the dissolved oxygen saturation DO value at 10%-30% using a speed agitator, and used vegetable oil as antifoam agent to control the foam. Samples were collected every 4 hours and feeding was provided as needed. The number and the morphology of the bacteria were examined under microscope. During the 0^{th}-22^{nd} hours, 1 g/L˜1.2 g/L of corn flour was supplemented at each time in accordance with the dose used in Example 2. Concentration of NH₄Cl was controlled at 0.5 g/L˜0.1 g/L, feeding was stopped towards the end of the log phase. The number of bacteria reached 2.2×10⁹ cfu/mL after 24 hours of fermentation.
5) Second stage (from the end of the log phase to the maturation of the endospores). Control parameters: temperature at 32-33° C. during 24^{th}-30^{th} hours, pH value in the range of 7.0˜7.5, air flow rate of 1:0.8˜1.2 (v/v·min), controlled the dissolved oxygen saturation DO value at 10%˜30% using a speed agitator. Supplemented with calcium carbonate at 0.5 g/L. Temperature was then changed to 37° C. and pH value adjusted to 8.0˜8.7. Culture was carried on until the end of fermentation (36 hours). The number of endospores reached 1.8×10⁹ cfu/mL after 36 hours of fermentation. Sporulation rate was 82%.

### Example 6

1. Activation of the Strain and Culture and Expansion of the Inoculum
1) Activation of the strain and culture of the slanting inoculum: Bacillus mucilaginosus HSCUP-76-8 was inoculated in the slanting culture medium for activation, with temperature controlled at 30° C., and cultured for 36 hours, thereby obtaining the original slanting inoculum. The original slanting inoculum was then inoculated in the slanting culture medium and cultured under the same prescribed conditions, thereby obtaining the slanting inoculum.
2) Production of shake-flask inoculum: The slanting inoculum was inoculated in a shake-flask medium with 100 ml medium per 250 ml shake-flask, cultured at 30° C., rpm 200 r/min for 12 hours, thereby obtaining a mother inoculum. The mother inoculum was inoculated in the shake-flake medium and cultured under the same prescribed conditions, thereby obtaining the liquid of shake-flask inoculum.
3) Culture of fermentation seed: Using seed fermentation medium 3, with charging coefficient of 0.7, steam sterilization under 121° C. for 20 minutes, the liquid of shake-flask inoculum was inoculated with an inoculum size of 5%, and cultured at 29° C., air flow rate of 1.0:0.8˜1.2 (v/v·min), dissolved oxygen saturation DO value controlled at 10%-30% using a speed agitator, pH value controlled at 7.2±0.2, for 12 hours, thereby obtaining a liquid inoculum of fermentation seed at log phase.

2. Two-Stage Fermentation Production
4) First stage: Using basal fermentation medium 7, with charging coefficient of 0.7, steam sterilization under 121° C. for 20 minutes. Inoculum size of 7%. Control parameters: temperature 30˜33° C., pH value of 7.2±0.2, air flow rate of 1:0.8˜1.2 (v/v·min) during the 0^{th}-26^{th} hours, controlled the dissolved oxygen saturation DO value at 10%-30% using a speed agitator, and used vegetable oil as antifoam agent to control the foam. Samples were collected every 4 hours and feeding was provided as needed. The number and the morphology of the bacteria were examined under microscope. 1 g/L˜1.4 g/L of corn flour was supplemented at each time during the 0^{th}-18^{th} hours; and 1.2 g/L˜1.4 g/L of corn flour was supplemented at each time during the 18^{th}-24^{th} hours. Concentration of ammoniacal nitrogen was controlled at 0.5 g/L˜0.1 g/L using NH₄Cl, feeding was stopped towards the end of the log phase. The number of bacteria reached 2.4×10⁹ cfu/mL after 26 hours of fermentation.
5) Second stage (from the end of the log phase to the maturation of the endospores). Control parameters: temperature at 32-33° C. during 26^{th}-32^{nd} hours, pH value in the range of 7.0˜7.5, air flow rate of 1:0.8˜1.2 (v/v·min), controlled the dissolved oxygen saturation DO value at 10%˜30% using a speed agitator. Supplemented with calcium carbonate at 0.5 g/L. Temperature was then changed to 37° C. and pH value adjusted to 8.0˜8.9. Culture was carried on until the end of fermentation (38 hours). The number of endospores reached 1.8×10⁹ cfu/mL after 36 hours of fermentation. Sporulation rate was 75%

### Example 7

1. Activation of the Strain and Culture and Expansion of the Inoculum
1) Activation of the strain and culture of the slanting inoculum: Bacillus mucilaginosus HSCUP-76-8 was inoculated in the slanting culture medium for activation, with temperature controlled at 30° C., and cultured for 36 hours, thereby obtaining the original slanting inoculum. The original slanting inoculum was then inoculated in the slanting culture medium and cultured under the same prescribed conditions, thereby obtaining the slanting inoculum.
2) Production of shake-flask inoculum: The slanting inoculum was inoculated in a shake-flask medium with 100 ml medium per 250 ml shake-flask, cultured at 30° C., rpm 200 r/min for 12 hours, thereby obtaining a mother inoculum. The mother inoculum was inoculated in the shake-flake medium and cultured under the same prescribed conditions, thereby obtaining the liquid of shake-flask inoculum.
3) Culture of fermentation seed: Using seed fermentation medium 3, with charging coefficient of 0.7, steam sterilization under 121° C. for 20 minutes, the liquid of shake-flask inoculum was inoculated with an inoculum size of 5%, and cultured at 29° C., air flow rate of 1.0:0.8˜1.2 (v/v·min), dissolved oxygen saturation DO value controlled at 10%-30% using a speed agitator, pH value controlled at 7.2±0.2, for 12 hours, thereby obtaining a liquid inoculum of fermentation seed at log phase.

2. Two-Stage Fermentation Production
4) First stage: Using basal fermentation medium 7, with charging coefficient of 0.7, steam sterilization under 121° C. for 20 minutes. Inoculum size of 10%. Control parameters: temperature 30˜33° C., pH value of 7.2±0.2, air flow rate of 1:0.8˜1.2 (v/v·min) during the 0^{th}-26^{th} hours, controlled the dissolved oxygen saturation DO value at 10%-30% using a speed agitator, and used vegetable oil as antifoam agent to control the foam. Samples were collected every 4 hours and feeding was provided as needed. The number and the morphology of the bacteria were examined under microscope. 1 g/L˜1.4 g/L of corn flour was supplemented at each time during the 0^{th}-18^{th} hours; and 1.2 g/L˜1.4 g/L of corn flour was supplemented at each time during the 18^{th}-24^{th} hours. Concentration of ammoniacal nitrogen was controlled at 0.5 g/L˜0.1 g/L using NH₄Cl, feeding was stopped towards the end of the log phase. The number of bacteria reached 2.4×10⁹ cfu/mL after 26 hours of fermentation.
5) Second stage (from the end of the log phase to the maturation of the endospores). Control parameters: temperature at 32-33° C. during 26^{th}-32^{nd} hours, pH value in the range of 7.0˜7.5, air flow rate of 1:0.8˜1.2 (v/v·min), controlled the dissolved oxygen saturation DO value at 10%˜30% using a speed agitator. Supplemented with calcium carbonate at 0.5 g/L. Temperature was then changed to 37° C. and pH value adjusted to 8.0˜8.9. Culture was carried on until the end of fermentation (38 hours). The number of endospores reached 2.0×10⁹ cfu/mL after 38 hours of fermentation. Sporulation rate was 83%

In addition, bacteria described in the above examples are used in the production of actual products according to the follow manufacturing procedures:

1. Mineral Selection

Ore selection: The ore selected from the mines are high in quality and free from impurity.

Humic acid: Over 60% of organic matters and over 40% humic acid.

Castor Meal: Over 50% of protein, over 13 of nitrogen, phosphorus and potassium, and over 80% of organic matters.

2. Comminution

Use T130 Raymond to grind the ore to powders (around 200 meshes).

Use T100 Raymond to grind the humic acid to powders (around 100 meshes).

Use ordinary grind to grind castor meal to powders (above 80 meshes).

3. Mixed Granulation

Mix the mineral powder, humic acid and castor meal in the ratio of 25:20:50 in a rotary drum granulator, granules are made under steam and water spray, dried and then sieved. Granules are then sprayed with bacteria and dried powders respectively. The granules are then encapsulated, tested, weighed and packaged for delivery.

FIG. 1 shows a schematic diagram of apparatus of the source preparation shop.

The above manufacturing procedure has the following details:

Raw materials are mixed in the prescribed ratio and input to the rotary drum granulator, apply 6 kg of steam spray under steam pressure, and 5 kg of water spray collectively for granulation. Due to higher viscosity of the mineral powder, granules can be made without addition of missionary binders. The granules are then dried until their water content falls below 5% in a drying drum of which the entrance reaches 300° C. The granules are then sieved, where granules smaller than 3 mm in diameter were returned for repeating the granulation process, and granules larger then in 3 mm diameter are passed to the cooling drum, cooled to 20° C. and sieved for the second time to remove granules larger than 4 mm which was crushed for a new round of granulation. Granules of 3-4 mm in diameter is input to an encapsulating machine and sprayed with 20 kg/T of bacteria and 15 kg/T of powders. Encapsulated granules are then tested for quality, package and delivered.

The present manufacturing procedure has the following advantages: precise formula (controlled by computer), free of error, materials are transported on a belt transport system throughout the entire process, and enhanced yield of granules with the use of water/vapour during the granulation.

Due to the presence of active bacteria, production of biological organic fertilizers previously used in the art usually involve two steps of drying and two steps of cooling with temperature controlled under 80° C. However, this approach may lead to a waste of time and labour, since one single step of drying is sufficient for drying the materials and raw materials are generally sterilized. Moreover, to implement the three steps of drying and two steps of cooling, the industrial setup would be very complex and lead to a waste of energy.

The present invention improve the production process in the art by reducing the three drying steps and two cooling steps to one drying step and one cooling step. The present invention reduces the use of apparatuses and saves energy. Since bacteria is added in the final step of granule production, the quality of the fertilizer products and viability of the microorganisms can be maintained.

In addition, the applicant provides below explanations and descriptions as to the technology described in the above examples:

Samples were taken from soils in the He-shun potash shale mining area, specifically from areas suffered from serious weathering. Bacteria were isolated from the samples, purified and classified in order to obtain native potassium-decomposing bacteria with a higher capability in potassium decomposition.

2.1 Materials and Methods

2.1.1 Major Source and Reference Strain

Mineral sample: soil and water samples from He-shun potash shale mine.

Reagent: LiCl, analytical grade, Tianjin Fengchuan Chemical Reagent Technology Co., Ltd.

Reference Strain: Bacillus mucilaginosus, purchased from China General Microbiological Culture Collection Center, code AS 1.231.

2.1.2 Instruments and Apparatuses

Low temperature shaker (Harbin Donglian Dianzi Jishu Kaifa Company Limited), electronic balance (Shanghai Huanao Scientific Trading Company Limited), stainless steel screen cloth (Zhejiang Shangyu Jinshu Bianzhichang), phase contrast microscope (Nanjing Milite Yiqi Yibiao Company Limited), digital camera (NIKON D40, Japan), thermostatic incubator (Shanghai Yuejin Yiliao Qijiechang), oscillator (Taicang Shiyan Shebeichang), super clean bench (DL-CJ-1N, Harbin Donglian Dianzi Jishu Kaifa Company Limited), digital display thermostatic water bath (Guohua Dianzi Company Limited), YX series portable pressure steam sterilizing pot (Jiangbin Binjiang Nedical Apparatus Company Limited) petri dish, spreading roll etc.

2.1.3 Medium
(1) Alexander Solid Medium (g/L): sucrose 5 g, NaH₂PO₄ 2 g, MgSO₄.7H₂O 0.5 g, FeCl₃ 0.005 g, CaCO₃ 0.1 g, agar 20 g, pH 7.2.
(2) Alexander Liquid Culture Medium: Alexander solid medium without agar
(3) Potash Shale Mineral Powder Liquid Culture Medium: sucrose 5 g, potash shale mineral powder 1 g (100 meshes), NaH₂PO₄ 1 g, MgSO₄.7H₂O 0.5 g, FeCl₃ 0.005 g, CaCO₃ 0.1 g, distilled water 1000 ml, pH value 7.2.
(4) Potash Shale Mineral Powder Solid Culture Medium: same composition of medium (3), with an addition of agar 20 g. The medium will be light gray transparent when made into plate, due to the presence of potash shale. The peripheral of the colony will become transparent because of the decomposing of the potash shale mineral powder during the growth of potassium-decomposing bacteria, and the size of the transparent circle is positively correlated to the potassium decomposing capability of the strain. Therefore, this solid medium can be used for initial screening of the potassium-decomposing bacteria.

2.1.4 Sampling Method
(1) Preparation Work: aseptic shovel spoon, aseptic Erlenmeyer flask, aseptic bag.
(2) Sampling: Collect soil and water samples from 8 points aseptically in the He-shun potash shale mine. Soil sample 1: 20 cm-depth soil from the vegetation root part on the hill slope of the potash shale; Soil sample 2: 5˜15 cm-depth water from the inner storage ditch of the terrace of the potash shale; Soil sample 3: ground surface inside the cave of the potash shale; Soil sample 4: wet soil from the bottom of the pebble, taken from the dry-out river bed of the stream channel; Soil sample 5: soybean land where the potash shale is highly differentiated; Soil sample 6: tomato land where the potash shale is highly differentiated; Water sample 1: bottom of a discarded well; Water sample 2: a small puddle in a marsh-like land.

2.1.5. Screening Method

2.1.5.1 Initial Screening

The objective of this research is to discover a strain which is capable of decomposing potash shale, in order to release potassium in mineralized forms. According to the constituent analysis, element potassium in the potash shale mainly exists in the form of silicate. Therefore, specific culture condition is required for selective culturing of silicate-decomposing bacteria. The following isolation method is designed based on the characteristics of said bacteria (i.e., capable of nitrogen fixation and sporulation):
(1) Prepare solution for dilution and isolation. (1) Aseptic physiological saline: prepare 1000 ml 0.9% NaCl solution and aliquot it into 100 m11250 ml Erlenmeyer flask. Add in cotton plug and sterilize. (2) Aseptic water: aliquot distilled water into 18×180 mm tubes (9 ml each). Add cotton plug and sterilize.
(2) Weigh 50 g of the sample and grind it to 100 meshes.
(3) Weigh 5 g of powder sample to a shake-flask containing aseptic physiological saline, and fully oscillate it for 30 seconds until it is uniformly mixed.
(4) Put the above-mentioned flake-shake into a water bath at 60° C. for 10˜15 minutes, in order to kill the bacterial trophozoite from the sample. Obtain the endospore suspension, and remove strains without sporulation.
(5) On a clean bench, prepare serial dilution of the endospore suspension with aseptic water in the magnitude of 10⁻², 10⁻³, 10⁻⁴, and 10⁻⁵. Pipette 0.1 ml from each of the 10⁻³, 10⁻⁴, and 10⁻⁵ dilutions, and spread the sample on a plate containing Alexander medium in triplicate. Incubate the plates in a thermostatic incubator for 2˜3 days at 32° C.
(6) Select the growing colonies on the Alexander solid medium and exclude those which are not capable of nitrogen fixation. Inoculate the selected colonies on the slanting surface, and assign numbers to these colonies.
(7) Inoculate the assigned strains in the Alexander liquid culture medium, and culture them in a shaker, and determine whether the strain is a pure strain by observing their growth stability under the microscope.
(8) Inoculate the pure, stably grown strains on solid medium for screening potassium-decomposing capability. Incubate the plate in a thermostatic incubator for 3 days at 32° C.
(9) Observe the size of the colonies and measure the potassium-decomposing circle. The size of the colony primarily shows the growth rate of the strain, while the size of the potassium-decomposing circle primarily shows the capability of decomposing the potash shale.
(10) Select and keep the strains.

2.1.5.2 Re-Screening
(1) Preparation of medium: potash shale mineral powder liquid culture medium is used.
(2) Preparation of bacteria: (1) Inoculate the bacteria selected from intimal screening into Alexander solid medium for activation. Culture for 48 hours with temperature controlled at 32° C., thereby obtaining the original slanting inoculum. Then inoculate the original slanting inoculum on Alexander solid medium and culture it under the same condition, to obtain the activated slanting inoculum. (2) Inoculate the activated slanting inoculum into the liquid culture medium, 50˜100 ml medium/250 ml shake-flask, temperature 32° C., rpm 190 r/min, culture on shaker for 8˜12 hours, thereby obtaining the mother inoculum. The mother inoculum is then inoculated in the shake-flask medium and culture it under the same prescribed condition, thereby obtaining the liquid of shake-flask inoculum.
(3) Inoculation and culture: In a 250 ml Erlenmeyer flask containing 95 ml of potash shale mineral powder liquid culture medium, inoculate the shake-flask inoculum at an inoculum size of 5% and culture it in a thermostatic shaker at 32° C. and 200 r/min for 10 days. Prepare a triplicate for each sample. A negative control and a positive control were set up respectively. In the negative control, the pasteurized shake-flask inoculum is inoculated in the potash shale mineral powder medium at the same inoculum size. In the positive control, the reference bacteria is inoculated in the potash shale mineral powder medium at the same inoculum size.
(4) Testing: Transfer the fermented liquid from the above fermenting bottles into the centrifugal tube, centrifuge at 8000 r/min, collect 4 ml of the supernatant and thoroughly mixed with equal volume of LiCl (6 mmol/L). Determine the amount of potassium using a frame spectrometer.
(5) Determine the optimal strain: According to the experimental data, select the strain which shows the highest potassium-decomposing capability as the starting strain for mutation.

2.1.6 the Determination and Preservation of the Bacteria

Characterize the bacterial strain according to “Bergey's Manual of Determinative Bacteriology”^{[52]} and with reference to “Common Bacteria System Identification Manual”. Preserve the strain.

2.1.7 Fermentation Experiment

2.1.7.1 Material and Medium
(1) Bacterial species: Bacillus mucilaginosus HSC
(2) Apparatus and material

Super clean bench, cold plasma-modifying device (Changzhou Xinqu Shitai Dengliziti Jishu Kaifa Company Limited, Type HD), thermostatic shaker (Harbin Donglian Dianzi Jishu Kaifa Company Limited, HZQ-C air bath oscillator), electric thermostatic incubator (Shanghai Yuejin Yiliao Qijiechang, type HH.B.II.420-S), BIOFLO 5 L-fermenter (New Brunswick Scientific E Edison, N.J., USA) etc.
(3) Medium

Slanting culture medium: sucrose 5 g, NaH₂PO₄ 1 g, MgSO₄.7H₂O 0.5 g, FeCl₃ 0.005 g, CaCO₃ 0.1 g, agar 23 g, distilled water 1000 ml, pH value 7.2.

Liquid culture medium: sucrose 10 g, NH₄Cl 1 g, NaH₂PO₄ 1 g, MgSO₄.7H₂O 0.5 g, FeCl₃ 0.005 g, CaCO₃ 0.3 g, distilled water 1000 ml, pH value 7.2.

Fermentation medium: sucrose 2 g, corn flour 10 g, NH₄Cl 1.5 g, yeast extract 0.5 g, NaH₂PO₄ 1.5 g, MgSO₄.7H₂O 0.5 g, FeCl₃ 0.005 g, CaCO₃ 0.5 g, MnSO₄ 0.5 g, distilled water 1000 ml, pH value 7.2.

2.1.7.2 Method
(1) Inoculate Bacillus mucilaginosus HSC in the slanting culture medium for activation, culture it at 32° C. for 48 hours to obtain the original slanting inoculum. Then inoculate the original slanting inoculum in the slanting culture medium and culture it under the same condition to produce slanting inoculum.
(2) Inoculate the slanting inoculum in liquid culture medium with 50-100 ml medium/250 ml shake-flake, culture it in a shaker at 32° C., rpm 190 r/min for 8-12 hours to obtain the mother inoculum. The mother inoculum is then inoculate in the seed fermentation culture medium, and cultured under the same conditions to obtain the shake-flask inoculum.
(3) Using seed fermentation medium, with charging coefficient as 0.7˜0.8, steam sterilization under 121° C. for 20 minutes, the shake-flask inoculum is inoculated with an inoculum size of 5-10% and cultured at 29-33° C., air flow rate of 1.0:0.8˜1.0 (v/v·min), dissolved oxygen saturation DO value controlled at 10%-30% using a speed agitator, pH value controlled at 7.2±0.2 until the end of fermentation.
(4) Measure the number of bacteria using plate counting method during the fermentation process, and plot the growth curve and sporulation curve.

2.2 Result and Analysis

2.2.1 Initial Screening
(1) As isolated from the samples obtained from 8 sampling spots in He-shun, 90 strains were able to grow on the Alexander solid medium and 29 strains of them were able to grow actively and remained stable during passage. The morphology of the colonies of these 29 strains are as follows:
   [6] colony producing black pigment, having bulge white surface
   [9] colony with walnut color
   [11] orange colony
   [13] colony with shiny surface
   [15] colony with orange red color
   [16] colony with shiny surface and brown color; colony with white color
   [25] colony with lacquer appearance
   [31] transparent colony
   [32] smooth colony with white color
   [34] circular colony with yellow color in the middle, and a white edge
   [35] colony with grayish green peripheral
   [38] rough surface without shininess
   [40] greenish brown colony with shininess surface
   [47] brown colony with shiny transparent surface
   [50] transparent colony
   [55] white colony
   [58] colony with transparency and brown color
   [60] non-transparent colony with grayish white color
   [65] smooth colony with agar-like transparency
   [69] non-transparent colony with light gray color
   [72] yellow colony with the production of pigment and white rough matter in the middle
   [76] gel-like and transparent colony
   [77] blue colony
   [81] smooth colony with grey color
   [82] red colony
   [85] colony with military green color
   [86] filamentous colony with blackish brown color
   [88] smooth and transparent colony
   [89] rough colony with white color and with matter in dark color in the middle
(2) Five strains out of the 29 strains could be continuously passed and show a larger potassium-decomposing circle on the solid potassium-decomposing medium. FIG. 2.1 shows some of the screenings:

The five strains are assigned identity A, B, C, D and E respectively, and their characteristics as to colonies and potassium decomposing circles are depicted in Table 2.1. The diagram and the table indicate that the five strains had a higher potassium-decomposing capability. They were inoculated on Alexander slant surface for temporary preservation.

TABLE 2.1
Colonies and potassium decomposing circles of
potassium-decomposing bacteria
isolated from the potash shale mining area

| | | Diameter of the |
|---|---|---|
| | | Potassium-Decomposing |
| Code of Strain | Diameter of Colony/mm | Circle/mm |
| A | 8 | 10 |
| B | 6 | 9 |
| C | 6 | 12 |
| D | 5 | 8 |
| E | 7 | 9 |

2.2.2 Re-Screening

The five strains obtained from initial screening were tested for their capability in decomposing potash shale, and the results are shown in FIG. 2.2.

As seen from the potassium-decomposing experiment with reference to the negative and positive controls, strains A, B and E showed a lower potassium-decomposing capability than the reference strain, while potassium-decomposing capability of strains C and D was significantly higher than that of the reference strain (strain C: 2.3 μg/ml, and strain D: 1.2 μg/ml). The data showed that strain C has a higher potassium-decomposing capability. Therefore, strain C was initially selected as a strain for the production of potash shale bacterial fertilizer, or for mutagenesis.

2.2.3 Characterization of Bacteria
(1) Morphology of the colony on the Alexander solid medium: As seen from the culture on potassium-decomposing medium, strain C grew rapidly on the Alexander solid medium, and the diameter of the largest colony reached 8˜10 mm. The colony has the following characteristics: smooth surface, translucent and colorless, circular shape, smooth edge and bulge upwards (FIG. 2.4), and the colony could not be easily picked by inoculation ring and appeared stringy.
(2) Morphology of the bacteria and endospores under the optical microscope: As observed under the microscope, the bacteria were in rod shape and with two blunt ends. The size of the bacteria are roughly (0.8˜1.0) μm×(2˜4) μm, (FIG. 2.3); the morphology of endospores was elliptical, with its central or sub-terminal as the sporulation site (FIG. 2.5). The bacteria produced rich and thick capsules in a nitrogen-free culture medium (FIG. 2.6).
(3) Physiological and biochemical assays: the above-mentioned bacteria and reference strain were tested using physiological and biochemical assays; results are shown in the below table.

TABLE 2.2
Comparison of physiological and biochemical
characteristics between strain C and
reference strain Bacillus mucilaginosus

| Physiological and | | |
|---|---|---|
| Biochemical Characteristics | Reference Strain AS 1.231 | Strain C |
| Starch | + | + |
| Tyrosine Hydrolysis | − | − |
| Phenylalanine Deaminase | − | − |
| Yolk Lecithinase | − | − |
| Reduction of Nitrate to | d | − |
| Nitrite | | |
| Catalase Determination | + | + |
| Lysozyme Resistance | ND | + |
| Gelatin Hydrolysis | − | − |
| Cultured in the Medium | − | − |
| Containing 10% NaCl | | |
| V.P Test | − | − |
| Indole Test | − | − |

Note:
Symbols used in the Table are defined in accordance with “Common Bacteria System Identification Manual”:
“+” means that at least 90% of strains are positive;
“−” means that at least 90% of strains are negative;
“d” means that 11~89% of strains are positive;
“ND” means undetermined.

According to “Bergey's Manual of Determinative Bacteriology” and “Common Bacteria System Identification Manual”, and based on the observed morphology of strain C and the results of physiological and biochemical assays obtained from strain C and reference strain Bacillus mucilaginosus, it is determined that strain C is a type of Bacillus mucilaginosus. The inventor has named strain C as Bacillus mucilaginosus HSC, and preserved the strain under −80° C. using glycerol-based ultra-low temperature storage method.

2.2.4 Batch Fermentation of HSC

Using the plate counting method, the number of bacteria and endospores during the HSC fermentation process were measured and the data were plotted as a growth curve and a sporulation curve (FIG. 2.7). It is observed that the concentration of the bacterial strain in the broth can reach 1.6×10⁹ cfu/ml at maximum, the final yield of the endospores was 1.2×10⁹ cfu/ml, and the sporulation rate was 75%.

Although Bacillus mucilaginosus HSC isolated from the potash shale mineral powder possesses properties in favor of the production and applications, the strain is a wild type and is considerably less competent in high-density fermentation as compared to existing strains of Bacillus mucilaginosus which have attained the highest density in fermentation. Therefore, mutagenesis experiments were conducted in an attempt to improve relevant properties of the Bacillus mucilaginosus HSC.

3.1 Materials and Methods

3.1.1 Bacteria: Bacteria HSC isolated from the potash shale mineral powder.

3.1.2 Major Instruments and Apparatuses

Super clean bench (DL-CJ-1N, Harbin Donglian, with 25 W ultraviolet lamp equipped), cold plasma-modifying apparatus (Changzhou Xinqu Shitai Dengliziti Jishu Kaifa Company Limited, Type HD), thermostatic shaker (Harbin Donglian Dianzi Jishu Kaifa Company Limited, HZQ-C air bath oscillator), electric thermostatic incubator (Shanghai Yuejin Yiliao Qijiechang, type HH.B.II.420-S), frame spectrometer (Shanghai Jingmi Kexue Yiqi Company Limited Fenxi Yiqi Zongchang, FP-640), BIOFLO 5 L-fermenter (New Brunswick Scientific E edison, N.J., USA) etc.

3.1.3 Medium

Potash Shale Mineral Powder Liquid Culture Medium: sucrose 5 g, potash shale mineral powder 1 g, NaH₂PO₄ 1 g, MgSO₄.7H₂O 0.5 g, FeCl₃ 0.005 g, CaCO₃ 0.1 g, distilled water 1000 ml, pH value 7.2.

Potash Shale Mineral Powder Solid Culture Medium: Add 23 g/L agar in the potash shale mineral powder liquid culture medium.

Slanting Culture Medium: sucrose 5 g, NaH₂PO₄ 1 g, MgSO₄.7H₂O 0.5 g, FeCl₃ 0.005 g,

CaCO₃ 0.1 g, agar 23 g, distilled water 1000 ml, pH value 7.2.

Liquid Culture Medium: sucrose 10 g, NH₄Cl 1 g, NaH₂PO₄ 1 g, MgSO₄.7H₂O 0.5 g, FeCl₃ 0.005 g, CaCO₃ 0.3 g, distilled water 1000 ml, pH value 7.2.

Fermentation Medium: refined corn flour 2 g, corn flour 10 g, NH₄Cl 1.5 g, yeast extract 0.5 g, NaH₂PO₄ 1.5 g, MgSO₄.7H₂O 0.5 g, FeCl₃ 0.005 g, CaCO₃ 0.5 g, MnSO₄ 0.5 g, distilled water 1000 ml, pH value 7.2.

3.1.4 Mutagenesis Method

3.1.4.1 Ultraviolet Mutagenesis

Preparation of bacterial suspension: Bacillus mucilaginosus HSC was activated in slanting culture medium for two times, and then in shake-flask liquid culture medium for two times. When Bacillus mucilaginosus HSC reached log phase of growth during the culture, centrifuged the sample at rpm 8000 r/min for 20 minutes, removed the supernatant and collected the residue bacterial solution for preparing the bacterial suspension using aseptic water. The bacterial concentration was adjusted to 10⁷˜10⁸ cfu/ml. The bacteria were stored for later uses.

Ultraviolet mutagenesis: The suspension was placed 20 cm under an ultraviolet lamp (power of 25 W, and wavelength of 254 nm) in an aseptic petri dish (5 cm diameter), and illuminated for different exposure time (0-15 min). Death rate of the bacteria was estimated using the plate counting method, and the exposure time which led to a 99.9% death rate was selected as the exposure dose in the ultraviolet mutagenesis experiment. After ultraviolet treatment, mutated bacterial strains were diluted and spread on the potash shale mineral powder solid culture plate. The plates were incubated in a thermostatic incubator at 32° C., in dark, for 2˜3 days. A negative control was set up using untreated HSC strain.

Initial screening: Colonies which showed typical Bacillus mucilaginosus characteristics, and showed a larger potassium-decomposing circle as compared to the negative control were selected. A total of 90 colonies were selected and assigned an identity from HSCU-1 to HSCU-90. The colonies were inoculated into the corresponding tube for slanting culture. Appearing time and size of the colonies were examined and recorded.

Re-screening: using potassium-decomposing capability as the criterion as determined by: In a 250 ml Erlenmeyer flask containing 95 ml of potash shale mineral powder liquid culture medium, bacterial suspension to be tested was inoculated with an inoculum size as 5%. A triplicate was prepared for each sample, a negative control which was inoculated with the same amount of pasteurized bacterial suspension was also set up. The cultures were cultured in a thermostatic shaker at 32° C. and 200 r/min for 10 days. 4 ml of the supernatant was collected after centrifugation, and mixed with equal volume of LiCl (6 mmol/L). The amount of potassium was then measured using a frame spectrometer.

Determination of genetic stability: Mutated strains were continuously passed for five times and each of the generations was tested in a fermentation experiment to compare the quantity of bacteria and formation of endospores of each generations and examine the stability of potassium-decomposing characteristics of the mutated strains. The starting strain for mutation was used as a control in this study.

3.1.4.2 Induced Mutagenesis by the Injection of the N¹⁴⁺ Ion Stream

Preparation of bacterial pellicle: Bacillus mucilaginosus HSCU-76 which underwent ultraviolet mutagenesis was activated in slanting culture medium for two times, and then in shake-flask liquid culture medium for two times. The culture was centrifuged at rpm 8000 r/min for 20 minutes, removed the supernatant and collected the residue bacterial solution for preparing the bacterial suspension using aseptic water. After adjusting the concentration to 10⁷˜10⁸ cfu/ml, lml of the bacterial suspension was taken and spread on an aseptic 9 cm-petri dish, dried aseptically and stored in dark thereafter^{[12]}.

Implantation with N¹⁴⁺ ion stream: A cold plasma-modifying device (Type HD) equipped with a radiofrequency power source was used, N¹⁴⁺ ions were implanted at a power of 50 keV. Bacterial pellicle was subject to the radiofrequency power source and low-energy N¹⁴⁺ ions were used as the implantation ions. Under a power of 50 W, plasma mutagenesis was performed for 0 s, 5 s, 10 s, 15 s, 20 s, 25 s, 30 s, 35 s, 40 s and 45 s respectively. Bacteria were washed off from the petri dish with aseptic water and subsequently spread on a plate (with slanting culture medium). The plate was incubated in a thermostatic incubation at 30° C. for 2-3 days. A control was set using untreated HSCU-76 strain.

Initial screening: As compared to the control, colonies from the tested groups which first appeared in the shape of hemisphere and showed typical characteristics were selected.

Re-screening: using potassium-decomposing capability as the criterion as described in the re-screening of ultraviolet mutagenesis.

Small-scale 5 L-fermenter trial: the starting bacterial strain and the 9 mutated bacterial strains were cultured using basal fermentation medium for the comparison of their fermentation cycles. The quantity of bacteria and the formation of endospores of the 9 mutated strains at different time points were compared.

Determination of genetic stability: The starting strain and the mutated strains were continuously passed for five times and each of the generations was tested and compared with respect to the quantity of bacteria and formation of endospores. Mutated strains were also examined for their properties relevant to industrial production and finally preserved.

3.2 Result and Analysis

3.2.1 Ultraviolet Mutagenesis of Bacillus mucilaginosus HSC

Bacillus mucilaginosus HSC was inoculated in the liquid culture medium and cultured for 8 hours.

Mutation dose experiment was then conducted using suspension prepared from bacteria at the log phase of growth. As shown in FIG. 3.1, death rate (death rate=difference in number of living bacteria per ml before and after exposure/number of living bacteria per ml before exposure) reached 99.9% when the exposure time was 13 minutes. This exposure time was selected for mutagenesis study since it was believed to result in a high mutation rate.
(1) Characterization of the mutated strains and selective breeding of bacteria. Using potash shale mineral powder solid medium as the plate selection medium after the ultraviolet mutagenesis, 100 mutated strains were selected in total. Based on the size of colony and the size of clear zones produced upon decomposition of mineral powder, 9 strains showed significant results; namely: HSCU-22, HSCU-29, HSCU-54, HSCU-69, HSCU-76, HSCU-78, HSCU-79, HSCU-85, HSCU-87.
(2) Determination of potassium-decomposing capability. The 9 mutated strains were inoculated in potash shale mineral powder liquid culture medium and cultured for 10 days. The content of soluble potassium in the culture medium was determined (Table 3.1). The results in Table 3.1 indicated that HSCU-76 has the highest number of bacteria of 1.1×10⁸ cfu/ml, and the highest leaching rate of potassium of 2.6 μg/ml. As compared to the control group HSC, there was no significant increase in the number of bacteria; but the potassium-decomposing capability of HSCU-76 showed a remarkable increase.

TABLE 3.1
Comparison of potassium-decomposing capability of different strains

| | Strains | | | | | | | | | |
|---|---|---|---|---|---|---|---|---|---|---|
| | HSCU- | HSCU- | HSCU- | HSCU- | HSCU- | HSCU- | HSCU- | HSCU- | HSCU- | |
| | 22 | 29 | 54 | 69 | 76 | 78 | 79 | 85 | 87 | HSC |
| Time | 10 | 10 | 10 | 10 | 10 | 10 | 10 | 10 | 10 | 10 |
| Number of | 1 | 1 | 1.1 | 1 | 1.1 | 1 | 0.9 | 1 | 1 | 1 |
| Bacteria | | | | | | | | | | |
| Content of | 2.4 | 2.4 | 2.4 | 2.4 | 2.6 | 2.4 | 2.3 | 2.3 | 2.4 | 2.4 |
| Potassium | | | | | | | | | | |

Note:
Unit for time: t;
Unit for number of bacteria: 1 × 10⁸ cfu/ml,
Unit for content of potassium: μg/ml.
The number of bacteria was estimated using the dilution plate counting method.

(3) Determination of genetic stability: To conduct a fermentation test, different generations of HSCU-76 were separately inoculated in fermentation culture medium in a 5 L-fermenter. Potassium-decomposing capability of each generation was determined (Table 3.2). The results indicated that, when being subject to the same culture medium and same conditions for metabolic control, all the tested generations of HSCU-76 exhibited a higher number of bacteria and endospores, and a higher potassium-decomposing capability, suggesting that HSCU-76 is genetically stable.

TABLE 3.2
Fermentation stability of mutated strains

| | | Generation 1 | Generation 2 | Generation 3 | Generation 4 | Generation 5 |
|---|---|---|---|---|---|---|
| Fermentation | Number of Bacteria | 9.3 | 8.9 | 10.0 | 9.0 | 9.5 |
| | Number of | 7 | 6.8 | 7.5 | 7.1 | 7 |
| | Endospores | | | | | |
| Shake Flask | Number of Bacteria | 1.1 | 1.0 | 1.0 | 1.1 | 1.0 |
| | Content of | 2.6 | 2.5 | 2.5 | 2.6 | 2.5 |
| | Potassium (μg/ml) | | | | | |

Note:
The number of bacteria was estimated using dilution plate counting method. The number of endospores was estimated using dilution plate counting method, after being boiled at 80° C. for 10 minutes.

3.2.2 Mutagenesis of Bacillus mucilaginosus HSCU-76 by the Implantation of N¹⁴⁺ Ion Streams

Mutagenesis by ion stream implantation is a physical and chemical effect, which is an integrated method for mutagenesis including chemically and physically induced mutagenesis.

The method can induce abnormal changes in the chromosomes and damage and break the phosphate bases of the DNA, thereby resulting in an alternation or deletion of the genetic material at the gene or molecular level and greatly increasing the frequency of mutation.
(1) Effects of implantation of N⁺ ion on the survival of the bacteria. The survival curve in FIG. 3.2 displays a “saddle shape”, showing the change in survival rate of the bacteria along with an increase of implantation does of N⁺ ions. Given that the positive mutation rate induced by N⁺ ions implantation increases at first and drops later, while the negative mutation rate increases gradually, drops temporarily after a certain point of the implantation time and then gradually increases, taking both the positive and negative rates into account and in view of the curve, 25 s and 38 s (where the positive rate was maximal) should be selected as the doses for inducing mutagenesis.
(2) Characterization of the mutated strains and selective breeding of bacteria. Using potash shale mineral powder solid medium as the plate selection medium after the ion stream mutagenesis, 8 strains were selected based on the morphology of the colony and the size of clear zones produced upon decomposition of mineral powder (with reference to the control group). The 8 strains were then assigned an identity from HSCU-76-1 to HSCU-76-8.
(3) Determination of potassium-decomposing capability. The 8 mutated strains were inoculated in potash shale mineral powder liquid culture medium and cultured for 10 days. The content of soluble potassium in the culture medium was determined (Table 3.3). The results in Table 3.3 indicated that HSCUP-76-8 has the highest number of bacteria of 1.6×10⁸ cfu/ml, and the highest leaching rate of potassium of 2.8 μg/ml. As compared to the control group HSCU-76, HSCU-76-8 did not show a significant increase in potassium-decomposing capability but a remarkable increase in the bacterial concentration, HSCU-76-8 may be further studied in a fermentation testing.

TABLE 3.3
Comparison of potassium-decomposing capability of different strains

| | Strain | | | | | | | | |
|---|---|---|---|---|---|---|---|---|---|
| | *-1 | *-2 | *-3 | *-4 | *-5 | *-6 | *-7 | *-8 | HSCU-76 |
| Time | 10 | 10 | 10 | 10 | 10 | 10 | 10 | 10 | 10 |
| Number of | 0.9 | 1.2 | 1.1 | 1.4 | 1.1 | 1 | 0.9 | 1.6 | 1.1 |
| Bacteria | | | | | | | | | |
| Content of | 2.2 | 2.6 | 2.5 | 2.7 | 2.4 | 2.3 | 2.2 | 2.8 | 2.4 |
| Potassium | | | | | | | | | |

Note:
Unit for time: t;
Unit for number of bacteria: 1 × 10⁸ cfu/ml;
Unit for content of potassium: μg/ml.
*-X means HSCUP-76-X

(4) Small-scale 5 L-fermeter trial and determination of genetic stability. To conduct a fermentation test, different generations of HSCUP-76-8 were separately inoculated in fermentation culture medium in a 5 L-fermenter. Potassium-decomposing capability of each generation was determined (Table 3.4). The results indicated that, (1) when being subject to the same culture medium and same conditions for metabolic control, the HSCUP-76-8 exhibited a significant higher number of bacteria and endospores as compared to the starting strain HSCU-76; and (2) when being subject to the same culture medium and same conditions for metabolic control, all the tested generations of HSCUP-76-8 exhibited a higher number of bacteria and endospores, and a higher potassium-decomposing capability, suggesting that HSCUP-76-8 is genetically stable.

TABLE 3.4
Fermentation Stability of Mutated Strains

| | | Generation 1 | Generation 2 | Generation 3 | Generation 4 | Generation 5 |
|---|---|---|---|---|---|---|
| Fermenter | Number of | 22.5 | 20 | 22 | 21 | 22 |
| | Bacteria | | | | | |
| | Number of | 18.6 | 16.6 | 18.3 | 17.9 | 18.5 |
| | Endospores | | | | | |
| Shake-Flask | Number of | 1.4 | 1.5 | 1.4 | 1.4 | 1.6 |
| | Bacteria | | | | | |
| | Content of | 2.7 | 2.7 | 2.6 | 2.7 | 2.8 |
| | Potassium | | | | | |
| | (μg/ml) | | | | | |

Note:
The tested strain was HSCUP-76-8.

This section describes the optimization of ingredients and their ratios of the culture media, and fermentation conditions for Bacillus mucilaginosus HSCUP-76-8, in order to establish a high density fermentation process applicable for the industrial production.

4.1 Materials and Methods

4.1.1 Bacteria

Mutated strain HSCUP-76-8, which has been identified and deposited in the China General Microbiological Culture Collection Center, and assigned the Accession No. CGMCC No. 8481.

4.1.2 Major Instruments and Apparatuses

Super clean bench (DL-CJ-1N, Harbin Donglian), cold plasma-modifying apparatus (Changzhou Xinqu Shitai Dengliziti Jishu Kaifa Company Limited, Type HD), electronic balance (Shanghai Huanao Scientific Trading Company Limited), thermostatic shaker (Harbin Donglian Dianzi Jishu Kaifa Company Limited, HZQ-C air bath oscillator), YX series portable pressure steam sterilizing pot (Jiangbin Binjiang Nedical Apparatus Company Limited), electric thermostatic incubator (Shanghai Yuejin Yiliao Qijiechang, type HH.B.II.420-S), BIOFLO 5 L-fermenter (New Brunswick Scientific E edison, N.J., USA), petri dish, spreader, aseptic shovel spoon, aseptic Erlenmeyer flask, etc.

4.1.3 Medium

Medium 1 (Slanting Culture Medium): sucrose 5 g, NaH₂PO₄ 1 g, MgSO₄.7H₂O 0.3˜0.7 g, FeCl₃ 0.005 g, CaCO₃ 0.1 g, agar 20 g, distilled water 1000 ml, pH value 7.2.

Medium 2 (Activation Liquid Culture Medium): sucrose 5 g, NaH₂PO₄ 1 g, MgSO₄.7H₂O 0.3˜0.7 g, FeCl₃ 0.005 g, CaCO₃ 0.1 g, distilled water 1000 ml, pH value 7.2.

Medium 3 (Liquid Culture Medium): sucrose 5 g, (NH₄)₂SO₄ 1 g, yeast extract 1 g, NaH₂PO₄ 1 g, MgSO₄.7H₂O 0.3 g, FeCl₃ 0.005 g, CaCO₃ 0.1 g, distilled water 1000 ml, pH value 7.2.

Basal Fermentation Medium: Depends on the experimental results.

4.1.4 Method

4.1.4.1 Preparation of Shake-Flask Inoculum
(1) Bacillus mucilaginosus HSC was inoculated in the slanting culture medium for activation, with temperature controlled at 32° C., and cultured for 48 hours, thereby obtaining the original slanting inoculum. The original slanting inoculum was then inoculated in the slanting culture medium and cultured under the same prescribed conditions, thereby obtaining the activated slanting inoculum.
(2) The slanting inoculum was inoculated in the activation liquid medium with 50-100 ml medium per 250 ml shake-flask, cultured at 32° C., rpm 190 r/min for 8-12 hours, thereby obtaining a mother inoculum. The mother inoculum was inoculated in the activation liquid medium and cultured under the same prescribed conditions, thereby obtaining the liquid of shake-flask inoculum.

4.1.4.2 Optimization of Formula of Culture Medium and Culture Conditions

(1) Single-Factor Experiments

(i) Single-Factor Experiment: Carbon Source

Carbon source is one of the five major factors for microbial metabolism. This single-factor experiment was conducted using carbon source commonly used by Bacillus mucilaginosus; namely sucrose, starch and corn flour. In each test, 5 g of carbon source was used while other ingredients of the culture medium were added at the same amount as in the liquid culture medium. Inoculum size was 5% and the fermentation conditions were the same. The maximum concentration of bacteria and the final number of endospores in the fermentation broth were determined by assaying various batches of the broth.

(ii) Single-Factor Experiment: Nitrogen Source

Nitrogen source is also one of the five major factors for microbial metabolism. This single-factor experiment was conducted using nitrogen source commonly used by Bacillus mucilaginosus; namely ammonium sulfate, peptone and soybean cake powder. In each test, 1 g of nitrogen source was used while other ingredients of the culture medium were added at the same amount as in the liquid culture medium. Inoculum size was 5% and the fermentation conditions were the same. The maximum concentration of bacteria and the final number of endospores in the fermentation broth were determined by assaying various batches of the broth.

(iii) Single-Factor Experiment: Growth Factor

Growth factors are trace organic matters that are essential for the growth and reproduction of microbes. This single-factor experiment tested the effects of growth factors by three settings: no addition, addition of bean sprout extract; and addition of yeast extract. 0.5 g of bean sprout extract or yeast extract was added while other ingredients of the culture medium were added at the same amount as in the liquid culture medium. Inoculum size was 5% and the fermentation conditions were the same. The maximum concentration of bacteria and the final number of endospores in the fermentation broth were determined by assaying various batches of the broth.

(iv) Single-Factor Experiment: Inorganic Phosphorus

Inorganic phosphorus has an important role in the life activity of microbes. This single-factor experiment tested the effects of inorganic phosphorus by three settings: no addition, addition of tricalcium phosphate; and addition of dipotassium phosphate. 1 g of tricalcium phosphate; or dipotassium phosphate was added while other ingredients of the culture medium were added at the same amount as in the liquid culture medium. Inoculum size was 5% and the fermentation conditions were the same. The maximum concentration of bacteria and the final number of endospores in the fermentation broth were determined by assaying various batches of the broth.

(v) Single-Factor Experiment: Temperature

Temperature can influence the speed of metabolic activity of microbes, thereby affecting the fermentation cycle. This single-factor experiment tested three temperatures 28° C., 32° C., and 36° C. Liquid culture medium was used, inoculum size was 5% and the fermentation conditions were the same. The maximum concentration of bacteria and the final number of endospores in the fermentation broth were determined by assaying various batches of the broth.

(vi) Single-Factor Experiment: pH Value

pH value is another factor affecting the fermentation of microbes. This single-factor experiment tested three pH values 7.0, 7.5, and 8.0. Liquid culture medium was used, inoculum size was 5% and the fermentation conditions were the same. The maximum concentration of bacteria and the final number of endospores in the fermentation broth were determined by assaying various batches of the broth.

(2) Combinatorial Tests
(i) To determine the major ingredients in the culture medium and part of the fermentation conditions for HSCUP-76-8, this experiments designed three combinatorial experiments with variations in the seven factors of carbon source, nitrogen source, growth factor, inorganic phosphorus, pH value, temperature, and dissolved oxygen. Bacteria was inoculated at a size of 5% and cultured for 36 hours, and the number of living bacteria was chosen as the test indicator. Table 4.1 depicts the design.

TABLE 4.1
L₁₈ (3⁷) Design of combinatorial experiment

| | Carbon | | Growth | | | | |
|---|---|---|---|---|---|---|---|
| | Source | Nitrogen | Factor | Inorganic | pH | Temperature | Dissolved |
| | (g/L) | Source (g/L) | (g/L) | Phosphorus | Value | (° C.) | Oxygen |
| Level 1 | Sucrose | Soybean | / | / | 7 | 28 | 100 ml/250 ml |
| | 10 | Cake 1 | | | | | |
| Level 2 | Corn | Ammonium | Yeast | KH₂PO₄ | 7.5 | 32 | 100 ml/250 ml |
| | Flour 10 | sulfate 1 | Extract | | | | |
| Level 3 | Starch 10 | Peptone 1 | Bean | 1 Ca₃(Po₄)₂ | 8.0 | 36 | 100 ml/250 ml |
| | | | Sprout | | | | |
| | | | Extract | | | | |
| | | | 200 | | | | |

(ii) To determine the ratio of ingredients of the culture medium for HSCUP-76-8, this experiments designed three levels of combinatorial experiments studying seven factors: corn flour, ammonium sulfate, yeast extract, K₂HPO₄, MgSO₄, temperature, and dissolved oxygen. Bacteria was inoculated at a size of 5% and cultured for 36 hours, and the number of living bacteria was chosen as the test indicator. Table 4.2 depicts the design.

TABLE 4.2
L₁₈ (3⁷) Design of combinatorial experiment

| | Corn | | Yeast | | | | |
|---|---|---|---|---|---|---|---|
| | Flour | Ammonium | Extract | | | Temperature | Dissolved |
| | (g/L) | Sulfate (g/L) | (g/L) | K₂HPO₄ | MgSO4 | (° C.) | Oxygen |
| Level 1 | 5 | 0.5 | 0.1 | 0.1 | 0.1 | 32 | 100 ml/250 ml |
| Level 2 | 10 | 1.0 | 0.5 | 0.5 | 0.4 | 32 | 100 ml/250 ml |
| Level 3 | 15 | 1.5 | 1.0 | 1.0 | 0.8 | 32 | 100 ml/250 ml |

4.1.5 Batch Fermentation

To examine the effects of ingredients of culture medium, and fermentation conditions in the single-factor experiments and combinatorial experiments, a batch fermentation experiment was designed for validation.
(1) Preparation of the fermentation culture medium: Medium optimized in section 4.1.4.2 was used for the preparation of 3.3 L fermentation medium.
(2) Filling of the Fermenter and Pasteurization: BIOFLO 5 L-fermenter was installed, pH electrodes and dissolved oxygen electrodes were tested. Fermentation medium was poured into the fermenter after checking and pasteurized.
(3) Fermentation: 1.8 L activated shake-flask inoculum was inoculated into the fermentation tank under flame. Fermentation begun.
(4) Sampling and testing: Broth was sampled every 2 hours. Testing included the number of bacteria in the broth, the number of endospores and the corresponding content of sugar, nitrogen and phosphorus. Tables and graphs were plotted based on the obtained data.
(5) Results were analysis and discussed.

4.1.6 Two-Stage Control and High-Density Fermentation

To obtain a higher number of endospores in the broth, and in light of the plotted graphs as to the number of bacteria and endospores during the batch fermentation, the batch fermentation was divided into two stages: the growth stage in which the quantity of bacteria is increased, and the sporulation stage in which the bacteria are converted to endospores, thus establishing a two-stage fermentation process.

(1) Design and Preparation of Medium

Activation liquid culture medium, basal fermentation medium and feeding medium were designed according to the consumption-related characteristics of sugar, nitrogen and phosphorus determined in the batch fermentation.

(2) Metabolic Control of the Two-Stage Fermentation

Change in the amount of ingredients in the fermentation medium and change in the pH value during the growth stage were monitored in a real-time manner, and measures were taken to reduce the lag phase and prolong the log phase so as to obtain a higher number of bacteria. In the sporulation stage, necessary measures were taken to promote the rapid conversion of bacteria into endospores.

4.1.7 Chemical Analysis
(1) Microscopic observation of the morphology of bacteria: to distinguish the morphology of bacteria at different phases;
(2) Sugar determination: use Anthrone reagent to determine the sugar content in the broth [53]
(3) Nitrogen determination: use indophenol blue spectrophotometry to determine the nitrogen content in the broth^{[54]}.
(4) Determination of number of bacteria and endospores: use the dilution plate spreading method.

4.2 Results and Analysis

4.2.1 Results of the Single-Factor Experiments

In all of the single-factor experiments, the units of the concentration of the bacteria and the number of endospores in the broth were 1×10⁸ cfu/ml. The data referred to the average of the numbers of bacteria and endospores obtained from the triplicate.

4.2.1.1 Result of Single-Factor Experiment: Carbon Source (FIG. 4.1)

The results of the experiment demonstrated that the number of bacteria (1×10⁸ cfu/ml) in the broth and the sporulation rate were different when different carbon sources were used. When sucrose was added as the carbon source, the number of bacteria was 0.8, while the number of endospores was 0.61 with a sporulation rate of 76%. When corn flour was added as the carbon source, the number of bacteria was 0.9, while the number of endospores was 0.67 with a sporulation rate of 74%. When starch was added as the carbon source, the number of bacteria was 0.85, while the number of endospores was 0.68 with a sporulation rate of 80%. 4.2.1.2 Result of single-factor experiment: Nitrogen source

The results of the experiment (FIG. 4.2) demonstrated that the number of bacteria in the broth (1×10⁸ cfu/ml) differed slightly when ammonium sulfate, peptone or soybean cake was used as the nitrogen sources, while the effects on sporulation were more significant.

The maximum amount of bacteria observed in the three types of the broth was around 0.9. When ammonium sulfate was added as the nitrogen source, the number of endospores was 0.68 with a sporulation rate of 76%. When peptone was added as the nitrogen source, the number of endospores was 0.55 with a sporulation rate of 61%. When soybean cake was added as the nitrogen source, the number of endospores was 0.57 with a sporulation rate of 68%. The results indicated that inorganic nitrogen sources leads to a higher sporulation rate is higher than organic nitrogen sources.

4.2.1.3 Result of Single-Factor Experiment: Growth Factors

The results of the experiment (FIG. 4.3) demonstrated that the number of bacteria (1×10⁸ cfu/ml) in the broth and the sporulation rate were different when different growth factors were used. When yeast extract was added as the growth factor, the average number of bacteria was 1 and the number of endospores was 0.78 with a sporulation rate of 78%. When bean sprout extract was added as the growth factor, the average number of bacteria was 0.8 and the number of endospores was 0.6 with a sporulation rate of 76%. The highest average number of bacteria in medium without the addition of growth factors was 0.75 and the number of endospores was 0.57 with a sporulation rate of 76%.

4.2.1.4 Result of Single-Factor Experiment: Inorganic Phosphorus

As shown in FIG. 4.4, there were no significant differences in the number of bacteria in the broth between tricalcium phosphate and dipotassium phosphate; both samples attained the highest number of around 0.9. When tricalcium phosphate was added as a growth factor, the final number of endospores in the broth was 0.7, with a sporulation rate of 79%. When dipotassium phosphate was added as a growth factor, the final number of endospores in the broth was 0.68, with a sporulation rate of 76%. In case where no inorganic salt was added, the highest number of bacteria in the broth was 0.5, the highest number of endospores in the broth was 0.35, with a sporulation rate of 75%.

4.2.1.5 Result of Single-Factor Experiment: pH Value

As shown in FIG. 4.5, the number of bacteria obtained from fermentation using media at different pH values was considerably different. Bacterial growth was better when the pH value was slightly alkaline. When pH value was 7.5, the highest concentration of the bacteria was 1. When pH value exceeded 8, the number of bacteria in the broth declined dramatically. pH values of 7, 7.5 and 8 were chosen for the combinatorial experiments.

4.2.1.6 Result of Single-Factor Experiment: Temperature

As shown in FIG. 4.6, when fermentation temperature was below 32° C., the number of bacteria increased gradually as the temperature increased, although the differences were not significant. However, in samples where the temperature exceeded 36° C., the number of bacteria dropped rapidly as the temperature increased. The number of bacteria in the broth was only 0.4 at 40° C. 28° C., 32° C., and 36° C. were chosen for the combinatorial experiments.

4.2.2 Result of Combinatorial Experiments

In the combinatorial experiments, the units of the concentration of the bacteria and the number of endospores in the broth were 1×10⁸ cfu/ml. The data referred to the average of the numbers of bacteria and endospores obtained from the triplicate. 4.2.1.1 Results of the first combinatorial experiment

Results of the first combinatorial experiment are shown in Table 4.3 and 4.4.

TABLE 4.3
L₁₈ (3⁷) Heuristic Analysis Table

| | Carbon | Nitrogen | Growth | | | | | |
|---|---|---|---|---|---|---|---|---|
| Testing | Source | Source | Factor | Inorganic | pH | Temperature | Dissolved | |
| factors | (g/L) | (g/L) | (g/L) | Salt | Value | (° C.) | Oxygen | Results |
| Trial 1 | 1 | 1 | 1 | 1 | 1 | 1 | 1 | 0.3 |
| Trial 2 | 1 | 2 | 2 | 2 | 2 | 2 | 2 | 0.8 |
| Trial 3 | 1 | 3 | 3 | 3 | 3 | 3 | 3 | 0.5 |
| Trial 4 | 2 | 1 | 1 | 2 | 2 | 3 | 3 | 1 |
| Trial 5 | 2 | 2 | 2 | 3 | 3 | 1 | 1 | 0.4 |
| Trial 6 | 2 | 3 | 3 | 1 | 1 | 2 | 2 | 0.8 |
| Trial 7 | 3 | 1 | 2 | 1 | 3 | 2 | 3 | 0.7 |
| Trial 8 | 3 | 2 | 3 | 2 | 1 | 3 | 1 | 1.4 |
| Trial 9 | 3 | 3 | 1 | 3 | 2 | 1 | 2 | 1.2 |
| Trial 10 | 1 | 1 | 3 | 3 | 2 | 2 | 1 | 0.7 |
| Trial 11 | 1 | 2 | 1 | 1 | 3 | 3 | 2 | 0.2 |
| Trial 12 | 1 | 3 | 2 | 2 | 1 | 1 | 3 | 1.1 |
| Trial 13 | 2 | 1 | 2 | 3 | 1 | 3 | 2 | 1.6 |
| Trial 14 | 2 | 2 | 3 | 1 | 2 | 1 | 3 | 1.3 |
| Trial 15 | 2 | 3 | 1 | 2 | 3 | 2 | 1 | 0.3 |
| Trial 16 | 3 | 1 | 3 | 2 | 3 | 1 | 2 | 0.2 |
| Trial 17 | 3 | 2 | 1 | 3 | 1 | 2 | 3 | 1.2 |
| Trial 18 | 3 | 3 | 2 | 1 | 2 | 3 | 1 | 0.5 |
| Average 1 | 0.600 | 0.750 | 0.700 | 0.633 | 1.067 | 0.750 | 0.600 | |
| Average 2 | 0.900 | 0.883 | 0.850 | 0.800 | 0.917 | 0.750 | 0.800 | |
| Average 3 | 0.867 | 0.733 | 0.817 | 0..933 | 0.383 | 0.633 | 0.967 | |
| Range | 0.300 | 0.150 | 0.150 | 0.300 | 0.684 | 0.117 | 0.367 | |

TABLE 4.4
L₁₈ (3⁷) Variance Analysis

| | Sum of | | | | |
|---|---|---|---|---|---|
| Testing | Squares of | Degree of | F | F Critical | Significance |
| factors | Deviations | Freedom | Proportion | Value | Level |
| Carbon | 0.324 | 2 | 0.823 | 3.740 | |
| Source | | | | | |
| Nitrogen | 0.081 | 2 | 0.206 | 3.740 | |
| Source | | | | | |
| Growth | 0.074 | 2 | 0.188 | 3.740 | |
| Factor | | | | | |
| Inorganic | 0.271 | 2 | 0.688 | 3.740 | |
| Salt | | | | | |
| pH Value | 1.548 | 2 | 3.932 | 3.740 | * |
| Temper- | 0.054 | 2 | 0.137 | 3.740 | |
| ature | | | | | |
| Dissolved | 0.404 | 2 | 1.026 | 3.740 | |
| Oxygen | | | | | |
| Error | 2.76 | 14 | | | |

From Table 4.4 “L₁₈ (3⁷) Variance Analysis”, it is found that results of pH value were significant when a=0.05. The difference in the average value of Levels 1 and 2 in the heuristic analysis table was not significant, and the pH 7.5 which is optimal to bacteria was selected. Results of the other testing factors were not significant.

From Table 4.3 “L₁₈ (3⁷) Heuristic Analysis”, it is determined that the effects of the testing factors on the degree of fermentation are in the following descending order: dissolved oxygen, carbon source, inorganic phosphorus, growth factor and nitrogen source. Based on the analysis, the following ingredients of culture medium and fermentation conditions were found to be optimal and selected: corn flour as the carbon source, ammonium sulfate as the nitrogen source, yeast extract as the growth factor, Ca₃(PO₄)₂ as the inorganic phosphorus, 32° C. as the temperature, and 7.5 as the pH value. For industrial production, Ca₃(PO₄)₂ is generally substituted by K₂HPO₄ for its higher price than K₂HPO₄,

4.2.1.1 Second Combinatorial Experiment

Results of the second combinatorial experiment are shown in Table 4.5 and 4.6.

TABLE 4.5
L₁₈ (3⁷) Data of combinatorial experiment

| Testing | Corn | Ammonium | Yeast | | | | Dissolved | |
|---|---|---|---|---|---|---|---|---|
| factors | Flour | Sulfate | Extract | K₂HPO₄ | MgSO₄ | Temperature | Oxygen | Results |
| Trial 1 | 1 | 1 | 1 | 1 | 1 | 1 | 1 | 0.4 |
| Trial 2 | 1 | 2 | 2 | 2 | 2 | 2 | 2 | 0.8 |
| Trial 3 | 1 | 3 | 3 | 3 | 3 | 3 | 3 | 0.6 |
| Trial 4 | 2 | 1 | 1 | 2 | 2 | 3 | 3 | 1 |
| Trial 5 | 2 | 2 | 2 | 3 | 3 | 1 | 1 | 1.4 |
| Trial 6 | 2 | 3 | 3 | 1 | 1 | 2 | 2 | 0.9 |
| Trial 7 | 3 | 1 | 2 | 1 | 3 | 2 | 3 | 0.4 |
| Trial 8 | 3 | 2 | 3 | 2 | 1 | 3 | 1 | 0.7 |
| Trial 9 | 3 | 3 | 1 | 3 | 2 | 1 | 2 | 0.9 |
| Trial 10 | 1 | 1 | 3 | 3 | 2 | 2 | 1 | 0.8 |
| Trial 11 | 1 | 2 | 1 | 1 | 3 | 3 | 2 | 0.4 |
| Trial 12 | 1 | 3 | 2 | 2 | 1 | 1 | 3 | 0.8 |
| Trial 13 | 2 | 1 | 2 | 3 | 1 | 3 | 2 | 1.6 |
| Trial 14 | 2 | 2 | 3 | 1 | 2 | 1 | 3 | 0.6 |
| Trial 15 | 2 | 3 | 1 | 2 | 3 | 2 | 1 | 0.7 |
| Trial 16 | 3 | 1 | 3 | 2 | 2 | 1 | 2 | 0.8 |
| Trial 17 | 3 | 2 | 1 | 3 | 1 | 2 | 3 | 1 |
| Trial 18 | 3 | 3 | 2 | 1 | 2 | 3 | 1 | 0.3 |
| Average 1 | 0.633 | 0.833 | 0.733 | 0.500 | 0.900 | 0.817 | 0.717 | |
| Average 2 | 1.033 | 0.817 | 0.883 | 0.800 | 0.733 | 0.767 | 0.900 | |
| Average 3 | 0.683 | 0.700 | 0.733 | 1.050 | 0.717 | 0.767 | 0.733 | |
| Range | 0.400 | 0.133 | 0.150 | 0.550 | 0.183 | 0.050 | 0.183 | |

TABLE 4.6
L₁₈ (3⁷) Variance Analysis

| | Sum of | | | F | |
|---|---|---|---|---|---|
| Testing | Squares of | Degree of | F | Critical | Significance |
| factors | Deviations | Freedom | Proportion | Value | Level |
| Corn Flour | 0.570 | 2 | 2.112 | 3.740 | |
| (g/L) | | | | | |
| Ammonium | 0.063 | 2 | 0.233 | 3.740 | |
| Sulfate (g/L) | | | | | |
| Yeast | 0.090 | 2 | 0.334 | 3.740 | |
| Extract | | | | | |
| (g/L) | | | | | |
| K₂HPO₄ | 0.910 | 2 | 3.372 | 3.740 | |
| MgSO₄ | 0.123 | 2 | 0.456 | 3.740 | |
| Temperature | 0.010 | 2 | 0.037 | 3.740 | |
| (° C.) | | | | | |
| Dissolved | 0.123 | 2 | 0.456 | 3.740 | |
| Oxygen | | | | | |
| Error | 1.89 | 14 | | | |

From Table 4.6 “L₁₈ (3⁷) Variance Analysis”, it is found that results of all the testing factors were insignificant when a=0.05.

From Table 4.5 “L₁₈ (3⁷) Heuristic Analysis”, it is determined that inorganic phosphorus and carbon source had the largest impact on the degree of fermentation. Based on the analysis, the following ingredients of culture medium (unit: g/L) and fermentation conditions were found to be optimal and selected: corn flour 10, ammonium sulfate 0.5, yeast extract 0.5, K₂HPO₄ 1, MgSO₄ 0.1, temperature at 32° C., and the pH value at 7.5.

4.2.3 Results of Batch Fermentation Experiment

Using the plate counting method, the number of bacteria and endospores during the fermentation process of HSC were measured and the data were plotted to prepare a growth curve and sporulation curve as shown in FIG. 4.7. It is observed that 0^{th}-10^{th} hour was the lag phase, 10^{th}-24^{th} hour was the log phase, and 24^{th}-48^{th} hour was the death phase. The highest concentration of the bacteria in the broth was 2.3×10⁹ cfu/ml, and the final yield of endospores was 1.9×10⁹ cfu/ml, with a sporulation rate of 82.6%.

As shown from the data on sugar content in the broth as measured by the anthrone method, and the data on ammoniacal nitrogen content in the broth as measured by the indophenol blue spectrophotometric method, there was a considerable increase in the sugar and ammoniacal nitrogen content during the lag phase. During the log phase, the total sugar content dropped rapidly, while the content of ammoniacal nitrogen decreased slightly. The results indicated that the bacteria were able to excrete enzymes out of the cells for decomposing the corn flour during the lag phase, thereby increasing the amount of sugar and nitrogen in the broth.

4.2.4 Design and Results of the Two-Stage High Density Fermentation

4.2.4.1 Design of the Two-Stage High Density Fermentation

According to the growth and metabolism patterns of the present bacterial strain, the fermentation process were divided into two stages for manipulation; namely the bacterial growth stage and the sporulation phase.
(1) First stage: (from inoculation to the end of log phase, with the objective of promoting bacterial growth and reproduction, to obtain the possible maximum number of bacteria): basal fermentation medium was designed as follows: corn flour 5 g, (NH)₂SO₄ 1˜2 g/NH₄Cl 0.5˜1 g, yeast extract 1˜2 g, NaH₂PO₄ 1˜2 g, MgSO₄.7H₂O 0.5˜1 g, FeCl₃ 0.005 g, distilled water 1000 ml, pH value 7.0˜7.4. Charging coefficient of 0.7-0.8, steam sterilization under 121° C. for 20 minutes, inoculum size of 5%˜10%. Control parameters: temperature 29˜33° C., pH value in the range of 7.0˜7.5, air flow rate of 1:0.8˜1.2 (v/v·min), controlling the dissolved oxygen saturation DO value at 20%-30% using a speed agitator, and using vegetable oil as antifoam agent to control the foam. Collect samples every 2 hours, then examine the number and morphology of the bacteria under microscope, and conduct chemical analysis on sugar and ammoniacal nitrogen. Promptly provide feeding according to the examination results, control sugar dose at 0.2 mg/L˜0.3 mg/L, and control nitrogen source at 0.2 mg/mL˜0.5 mg/mL using NH₄Cl, then stop providing additional sugar and nitrogen towards the end of the log phase.
(2) Second stage (from the end of the log phase to the maturation of the endospores, with the objective of sporulation with the possible maximum sporulation rate), control parameters: temperature at 33-37° C., pH value in the range of 7.2˜8.9, air flow rate of 1:1.0˜0.8 (v/v·min), controlling the dissolved oxygen saturation in DO value at 5%-10% using a speed agitator. When the bacterial growth is close to the stationary phase, add calcium carbonate to promote sporulation. Stop fermentation, and store the product when endospores occupy the entire visual field under the microscopic examination.

4.2.4.2 Control of Parameters and Results of Two-Stage High Density Fermentation
(1) Control of Parameters: The graph in FIG. 4.8 shows the bacteria growth and sporulation during the two-stage high density fermentation using a 5 L-fermenter. Basal fermentation medium was used in the second stage of the liquid shake-flask activation which significantly shortened the lag phase. Feeding of 5 g of corn flour was provided at 12^{th} hr, 14^{th} hr and 16^{th} hr respectively, and refined corn flour was added at 18^{th} hr, 20^{th} hr and 22^{th} hr. Temperature was controlled at 29° C. during 0^{th}˜9^{th} hr, and 31° C. during 9^{th}˜20^{th} hr; pH was controlled at 7.2±0.1; 32° C. during 20^{th}-24^{th} hr, dissolved oxygen at 0.8 h:1 (v/v·min) during 0^{th}˜9^{th}hr, and at 1:1 (v/v·min) during 9^{th}-24^{th} hr. When the bacteria was about to reach the stationary phase as assessed by their morphology under the microscope, the second stage manipulation was implemented as follows: 0.5 g calcium carbonate was added at 24^{th} hr, temperature was controlled at 34° C. during 24^{th}-48^{th} hr; pH value was adjusted to 7.5. The pH value continuously increased along with the release of endospores from the bacteria, and finally reached 8.4 at the end of fermentation. Airflow was set at 1:1 (v/v·min) during 24^{th}˜32^{th} hr, and 0.8:1 (v/v·min) during 32^{th}˜48^{th}. The broth was examined at 48^{th} hr, and fermentation was stopped when endospores occupied the entire visual field under the microscopic examination.
(2) Result: the number of bacteria in the broth reached 2.8×10⁹ cfu/mL at maximum, and the final number of endospores in broth was 2.3×10⁹ cfu/mL, with a sporulation rate of 82%.

What is claimed is:

1. A Bacillus mucilaginosus, with culture collection no. as CGMCC No. 8481.

2. A two-stage fermentation process of producing Bacillus mucilaginosus, comprising the following steps:
(1) Inoculating Bacillus mucilaginosus HSCUP-76-8 on a slanting culture medium for activation, with temperature controlled at 29-33° C., culturing for 24-48 hours to obtain an original slanting inoculum, then inoculating the original slanting inoculum on the slant medium and culturing under the same condition to obtain a slanting inoculum;
(2) Inoculating said slanting inoculum in a liquid culture medium with 50-100 ml medium per 250 ml shake flake, culturing at temperature 29-33° C., rpm 160-220 r/min, for 8-12 hours on a shake bed, to obtain a mother inoculum; then inoculating the mother inoculum in a shake-flask medium, and culturing under the same condition, to obtain a liquid of shake-flask inoculum;
(3) Culturing using a seed fermentation medium, with charging coefficient of 0.7-0.8, steam sterilization under 121° C. for 20 minutes, inoculum size of 5%-10%, culturing temperature of 29˜33° C., air flow of 1:0.8˜1.0 (v/v·min), controlling the dissolved oxygen saturation DO value at 10%˜30% using a speed agitator, pH value controlled in the range of 7.2±0.2, culturing for 8-12 hours, to obtain a liquid inoculum of fermentation seed at log phase;
(4) First stage fermentation culturing, using basal fermentation medium, with charging coefficient of 0.7-0.8, steam sterilization under 121° C. for 20 minutes, inoculum size of 5%-10%; temperature at 29˜33° C., pH value controlled in the range of 7.0-7.2, air flow 1:0.8˜1.2 (v/v·min), controlling the dissolved oxygen saturation DO value at 20%˜30% using a speed agitator, and using vegetable oil as antifoam agent for the controlling of the foam; sampling in every 4 hours, then examining the number and morphologies of bacteria under a microscope, and conducting chemical analysis of sugar and ammoniacal nitrogen; ensuring appropriate feeding according to the examination result to control sugar dose at 2 g/L˜3 g/L, and controlling nitrogen source with NH₄Cl at 0.5 g/L˜0.1 g/L, then stopping the nitrogen supply towards the end of the log phase;
(5) Second stage fermentation culturing at temperature 33-37° C., pH value in the range of 7.2-8.9, airflow 1:1.0˜0.8 (v/v·min), controlling the dissolved oxygen saturation DO value as 5%˜10% using a speed agitator, then adding calcium carbonate to promote sporulation; terminating fermentation when endospores occupy the entire visual field under microscopic examination, and storing for later usage.

3. The process of claim 2, wherein said slanting culture media contains sucrose 5 g, NaH₂PO₄ 1 g, MgSO₄.7H₂O 0.5 g, FeCl₃ 0.005 g, CaCO₃ 0˜0.1 g, agar 20˜30 g, distilled water 1000 ml, pH value 7.0˜7.4.

4. The process of claim 2 wherein said seed liquid culture medium contains sucrose 5˜10 g, NH₄Cl 0.5˜1 g, NaH₂PO₄ 1˜1.5 g, MgSO₄.7H₂O 0.5˜1 g, FeCl₃ 0.005 g, CaCO₃ 0.1˜0.3 g, distilled water 1000 ml, pH value 7.0˜7.4.

5. The process of claim 2, wherein said seed fermentation medium contains sucrose 2˜5 g, starch 3˜10 g, (NH₄)₂SO₄ 1˜2 g, NH₄Cl 0.5˜1 g, yeast extract 1˜2 g, NaH₂PO₄ 1˜1.5 g, MgSO₄.7H₂O 0.5˜1 g, FeCl₃ 0.005 g, distilled water 1000 ml, pH value 7.0˜7.4.

6. The process of claim 2, wherein said basal fermentation medium contains sucrose 5 g, (NH₄)₂SO₄ 1˜2 g/NH₄Cl 0.5˜1 g, yeast extract 1˜2 g, NaH₂PO₄ 1˜2 g, MgSO₄.7H₂O 0.5˜1 g, FeCl₃ 0.005 g, distilled water 1000 ml, pH value 7.07.4.

7. The process of claim 2, where the sucrose is substituted with either corn flour or starch.

8. The process of claim 2, wherein the sucrose is substituted with a mixture of sucrose and corn flour.
